(19) United States
(12) Patent Application Publication
(10) Pub. No.: US 2023/0348358 A1
(43) Pub. Date: Nov. 2, 2023

(54) Serotonin Receptor Modulators

(71) Applicant: Northeastern University, Boston, MA (US)

(72) Inventor: Raymond BOOTH, Arlington, MA (US)

(21) Appl. No.: 18/020,451

(22) PCT Filed: Aug. 19, 2021
(86) PCT No.: PCT/US2021/046637

§ 371 Date: Feb. 9, 2023

Related U.S. Application Data
(60) Provisional application No. 63/067,853, filed on Aug. 19, 2020.

(51) Int. Cl. C07C 211/42; C07D 217/02; C07D 307/52; C07D 207/335; C07D 295/073; A61P 25/08

(52) U.S. Cl. CPC C07C 211/42; C07D 217/02; C07D 307/52; C07D 207/335; C07D 295/073; A61P 25/08; C07B 2200/07; C07C 2602/10; C07C 2601/08

(57) ABSTRACT

Compounds, compositions, and methods are provided for dual partial agonism of serotonin 5-HTS and 5-HTIA receptors. The enantiomerically pure 5-phenyl-2-aminotetralin (5-PAT) compounds can be used to treat or prevent substance use disorder, opioid use disorder, addiction, anxiety, psychosis, depression, autism spectrum disorder, fragile X syndrome, neurological disorders, neuropsychiatric disorders, repetitive behaviors, movement disorders, compulsions, tics, pain disorders, vasospastic disorders, migraine headache, seizures, epilepsy, social anxiety, addiction withdrawal, drug withdrawal, drug abuse, alcoholism, eating disorders, general inflammation disorders, miosis, inflammatory bowel disease, ulcerative colitis, Crohn's disease, and gastrointestinal disorders.

### CROSS-REFERENCE TO RELATED APPLICATIONS

This application claims priority to U.S. Provisional Application No. 63/067,853, filed 19 Aug. 2020, which is incorporated by reference herein in its entirety.

### STATEMENT REGARDING FEDERALLY SPONSORED RESEARCH OR DEVELOPMENT

This invention was made with government support under Grant Numbers DA047130 awarded by the National Institutes of Health, and W81XWH-15-1-0247 and W81XWH-17-1-0322 awarded by the Department of Defense. The government has certain rights in the invention.

### BACKGROUND

Drug addiction, also called substance use disorder (SUD), is a disease that can affect behavior and can lead to an inability to control the use of a legal or illegal drug. Prescription drug abuse is a major public health concern, and substances such as alcohol, opioids, marijuana, and nicotine are common drugs of abuse.

Serotonin receptors or 5-hydroxytryptamine (5-HT) receptors come in several classes and are involved in both drug use and in withdrawal symptoms. As knowledge of serotonin receptors has grown, different classes of receptors have been found to be expressed in various locations and to have different functions. Studies have linked alterations in central serotonergic systems to a wide range of mental and behavioral disorders, including SUD. No FDA-approved drugs specifically address psychiatric symptoms associated with withdrawal from opioids or other prescription drugs. While treatment of SUD can be carried out with benzodiazepine anxiolytics such as alprazolam, which targets GABA_{A} receptors, the treatment can cause sedation, the treatment compounds are themselves addictive, and the treatment often contributes to opioid overdoses. For example, the National Institute on Drug Abuse reports >30% of opioid overdoses also involve benzodiazepines (drugabuse.gov, March 2018). Selective serotonin reuptake inhibitors (SSRIs) which target the serotonin transporter (SERT) can alleviate anxiety and depression, but relief can take weeks, limiting utility for treating acute symptoms of drug withdrawal. Side effects from SSRI treatments are also prevalent. The numerous side-effects of SSRIs likely result from indiscriminate elevation of 5-HT levels during treatment, which can have non-therapeutic interactions at multiple 5-HT receptor types. While knowledge of various 5-HT receptors has grown, what is urgently needed is a correspondingly selective targeting of distinct 5-HT receptors to treat SUD and other disorders.

### SUMMARY

The present technology provides compounds and methods of treating diseases or disorders including neuropsychiatric disorders. Symptoms treated can be manifested as psychiatric symptoms or as physical symptoms. For example, the compounds and methods can be used to improve treatments for SUD by making the psychiatric symptoms from withdrawal a better patient alternative than the continued substance dependence. By selectively targeting distinct serotonin receptor types, the compounds and methods described herein can minimize adverse symptoms without significantly decreasing desirable behaviors.

The present technology can be further summarized by the following list of features.
1. A compound of Formula (I)

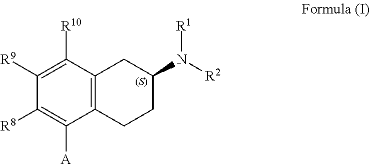

wherein A is selected from the group consisting of:

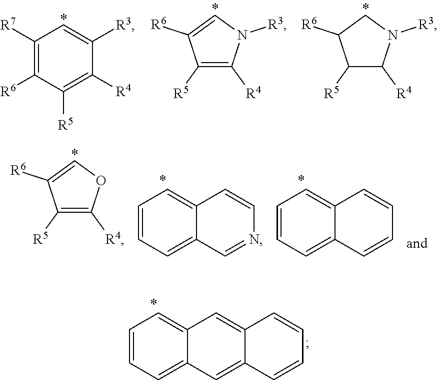

wherein A is attached to Formula (I) through carbon atom *;
wherein each of R¹ and R² is independently hydrogen or alkyl; and R¹ and R² may come together to form a substituted or unsubstituted alkyl ring, heterocyclic ring, or aromatic ring;
each of R³, R⁴, R⁵, R⁶, R⁷, R⁸, R⁹, and R¹⁹ is independently hydrogen, halo, hydroxy, acyl, acyloxy, alkyl, heteroalkyl, alkenyl, heteroalkenyl, alkynyl, heteroalkynyl, alkoxycarbonyl, carbonyl, cyano, sulfonamide, trifluoromethyl, trifluoromethoxy, nitro, amino, amido, and a cycloalkyl or cycloaryl, and wherein any two adjacent R groups may optionally come together to form a substituted or unsubstituted carbocyclic, aromatic, naphthalene, isoquinoline, or heterocyclic ring or ring system.
2. A compound selected from the group consisting of:

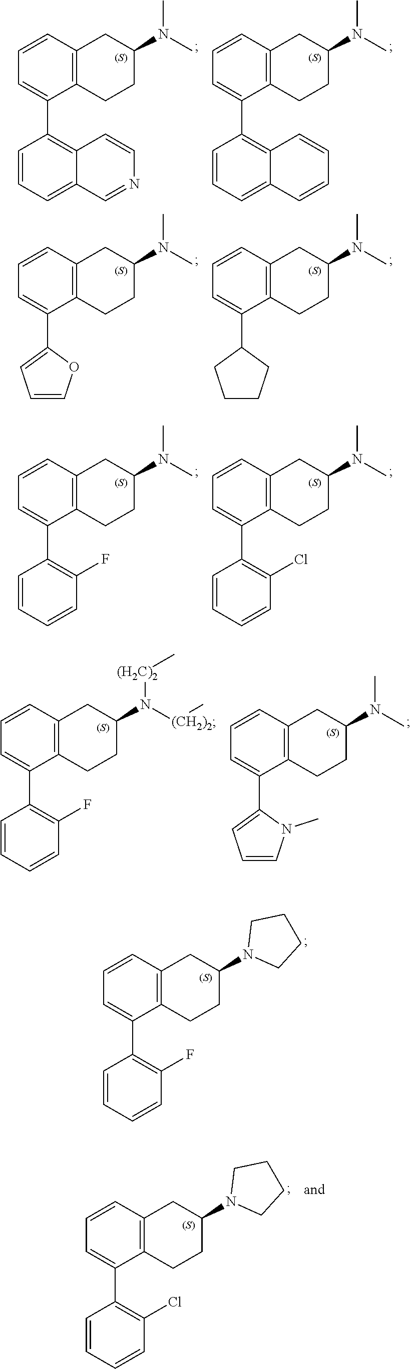

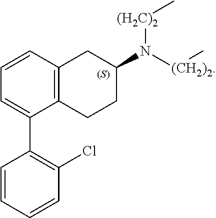

3. A pharmaceutical composition comprising a compound of claim 1 or claim 2, or a pharmaceutically acceptable salt, hydrate, or solvate thereof.
4. The pharmaceutical composition of claim 3, wherein the composition comprises at least about 70%, or at least about 80%, or at least about 85%, or at least about 90%, or at least about 95%, or at least about 97%, or at least about 99% of a single enantiomer of the compound.
5. The pharmaceutical composition of claim 3 or claim 4, wherein the compound binds to a serotonin 5-HT₇ receptor and/or a serotonin 5-HT_{1A} receptor with an affinity of less than about 100 nM, or less than about 50 nM, or less than about 25 nM, or less than about 20 nM, or less than about 10 nM, or less than about 5 nM, or less than about 2 nM, or less than about 1 nM.
6. The pharmaceutical composition of any one of claims 3-5, wherein the compound is a dual partial agonist of the serotonin 5-HT₇ and serotonin 5-HT_{1A} receptors.
7. The pharmaceutical composition of any one of claims 3-6, wherein the compound is an antagonist of a serotonin 5HT_{2B} receptor and/or a moderate affinity agonist of a serotonin 5HT_{2C} receptor.
8. The pharmaceutical composition of any one of claims 3-7, wherein the compound binds to the serotonin 5-HT_{2B} receptor with a binding affinity of less than about 100 nM.
9. The pharmaceutical composition of any one of claims 3-8, wherein the compound binds the serotonin 5-HT_{2A} receptor or the 5-HT_{2C} receptor with an affinity of greater than about 300 nM, or greater than about 400 nM, or greater than about 500 nM, or greater than about 750 nM, or greater than about 1 μM.
10. The pharmaceutical composition of any one of claims 3-9, wherein the compound binds one or more of the serotonin 5-HT₇ and 5-HT_{1A} receptors with at least about 10-fold, or at least about 20-fold, or at least about 30-fold, or at least about 40-fold, or at least about 50-fold, or at least about 75-fold, or at least about 100-fold higher affinity than the affinity with which it binds to either the serotonin 5-HT_{2A} receptor or the serotonin 5-HT_{2C} receptor.
11. A method of treating or preventing a disease or disorder, comprising administering a therapeutically effective amount of an at least 70% pure (S)-enantiomer dual partial agonist at the serotonin 5-HT₇ and 5-HT_{1A} receptors, the dual partial agonist having the structure of Formula (I):

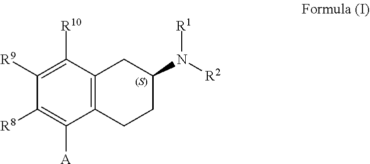

wherein A is selected from the group consisting of:

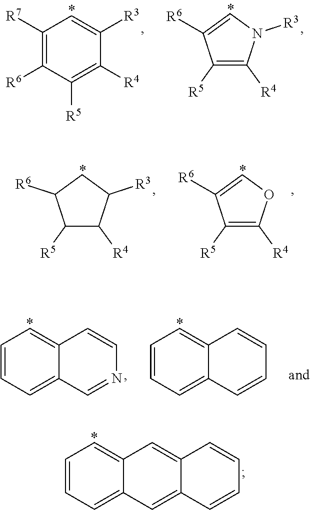

wherein A is attached to Formula (I) through carbon atom *;
wherein each of R¹ and R² is independently hydrogen or alkyl; and R¹ and R² may come together to form a substituted or unsubstituted alkyl ring, heterocyclic ring, or aromatic ring;
each of R³, R⁴, R⁵, R⁶, R⁷, R⁸, R⁹, and R¹⁹ is independently hydrogen, halo, hydroxy, acyl, acyloxy, alkyl, heteroalkyl, alkenyl, heteroalkenyl, alkynyl, heteroalkynyl, alkoxycarbonyl, carbonyl, cyano, sulfonamide, trifluoromethyl, trifluoromethoxy, nitro, amino, amido, and a cycloalkyl or cycloaryl, and wherein any two adjacent R groups may optionally come together to form a substituted or unsubstituted carbocyclic, aromatic, naphthalene, isoquinoline, or heterocyclic ring or ring system;
or a pharmaceutically acceptable salt, hydrate, or solvate thereof.
12. The method of claim 11, wherein the disease or disorder is selected from the group consisting of substance use disorder, opioid use disorder, addiction, anxiety, psychosis, depression, autism spectrum disorder, fragile X syndrome, neurological disorders, neuropsychiatric disorders, repetitive behaviors, movement disorders, compulsions, tics, pain disorders, vasospastic disorders, migraine headache, seizures, epilepsy, social anxiety, addiction withdrawal, drug withdrawal, drug abuse, alcoholism, eating disorders, general inflammation disorders, miosis, inflammatory bowel disease, ulcerative colitis, Crohn's disease, and gastrointestinal disorders.
13. The method of claim 11 or claim 12, wherein the disorder is substance use disorder caused by use of an opioid or THC.
14. The method of any one of claims 11-13, wherein the dual partial agonist comprises at least about 70%, or at least about 80%, or at least about 85%, or at least about 90%, or at least about 95%, or at least about 97%, or at least about 99% of a single enantiomer of Formula (I).
15. The method of any one of claims 11-14, wherein the dual partial agonist binds the serotonin 5-HT₇ and/or 5-HT_{1A} receptors with a binding affinity of less than about 100 nM, or less than about 50 nM, or less than about 25 nM, or less than about 20 nM, or less than about 10 nM, or less than about 5 nM, or less than about 2 nM, or less than about 1 nM.
16. The method of any one of claims 11-15, wherein the dual partial agonist is an antagonist at the serotonin 5HT_{2B} receptor and/or a moderate affinity agonist at the serotonin 5HT₂C receptor.
17. The method of any one of claims 11-16, wherein the dual partial agonist binds to the serotonin 5-HT_{2B} receptor with a binding affinity of less than about 100 nM.
18. The method of claim 17, wherein the dual partial agonist binds the serotonin 5-HT_{2A} and/or 5-HT_{2C} receptors with an affinity of greater than about 300 nM, or greater than about 400 nM, or greater than about 500 nM, or greater than about 750 nM, or greater than about 1 μM.
19. The method of any one of claims 11-18, wherein the dual partial agonist binds one or more of the serotonin 5-HT₇ and 5-HT_{1A} receptors with at least about 10-fold, or at least about 20-fold, or at least about 30-fold, or at least about 40-fold, or at least about 50-fold, or at least about 75-fold, or at least about 100-fold higher affinity than the affinity with which it binds to either the serotonin 5-HT_{2A} receptor or the serotonin 5-HT_{2C} receptor.
20. The method of any one of claims 11-19, wherein the dual partial agonist is a compound selected from the group consisting of:

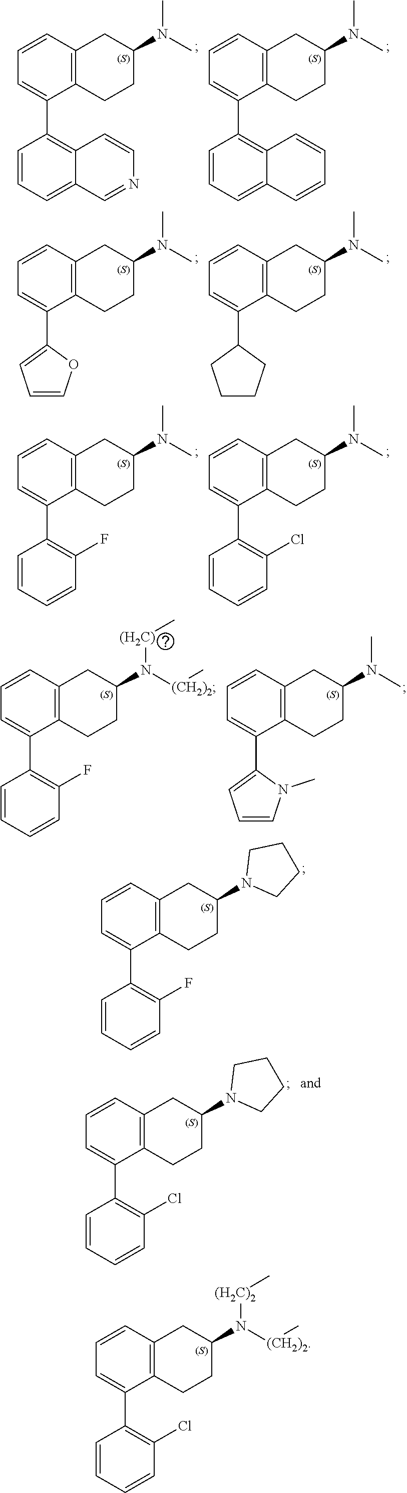

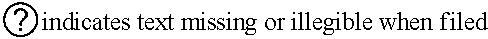

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1 shows the 5-phenyl-2-aminotetralin (5-PAT) moiety at top and examples of various 5-phenyl-2-aminotetralin (5-PAT) compounds.

FIG. 2 shows results of measured (S)-5-PAT scaffold yields as 5HT_{1A} agonist ligands. Buspirone, which is a known serotonin 5-HT_{1A} receptor partial agonist, is shown for comparison.

FIG. 3 shows results of measured (S)-5-PAT scaffold yields with various function as 5HT₇ ligands. AS-19 (a 5HT₇ agonist) and clozapine are shown for comparison.

FIG. 4 shows results of (S)-5-FPT, dose dependent testing for idiopathic repetitive jumping in 058/J mice.

FIG. 5 shows results of (S)-5-FPT testing for effects on (±)-DOI (5HT₂ agonist) elicited head-twitching in C57BL/6J mice. Buspirone (Busp 1) effects are shown for comparison.

FIG. 6 shows results of (S)-5-FPT testing for effects on MK801 (NMDA antagonist) elicited circling in C57BL/6J mice. Buspirone (Busp 1) effects are shown for comparison.

FIG. 7 shows results of (S)-5-FPT testing for effects on marble burying in FVB mice, which is an anxious and hyperactive WT strain.

FIG. 8 shows results of (S)-5-FPT versus vehicle for effects on locomotor behavior in three mouse models (C57BL/6J, FVB, and 058/J).

FIG. 9 shows results of (S)-5-FPT versus vehicle on social interactions in two different mouse strains.

FIG. 10A shows results of molecular docking at the 5HT_{1A} receptor for the (R) and (S) 5-FPT enantiomers. Both enantiomers are overlaid in the docking site for comparison. FIG. 10B shows results of molecular docking at the 5HT₇ receptor for the (R) and (S) 5-FPT enantiomers.

FIG. 11A shows docking of (2S)-5-PyT at the 5HT_{1A} receptor, where it is an agonist.

FIG. 11B shows (2S)-5-PyT docked at the 5HT₇ receptor, where it is an inverse agonist.

FIG. 12 shows results of molecular dynamics simulations for 5-PyT at the 5HT1A receptor (5-PyT-5HT1A) and for 5-PyT at the 5HT7 receptor (5-PyT-5HT7).

FIG. 13 shows percent lethal seizure, non-lethal seizure, wild-running and jumping (WRJ), and normal behavior of juvenile Fmr1 knockout mice exposed to high-decibel, high-frequency noise after administration of vehicle (left) or administration of (S)-5-FPT (right).

FIG. 14 shows c-Fos expression in dorsal hippocampus (CA1), basolateral amygdala (BLA), and lateral hypothalamus (HYP) of an adult, male FVB mouse after an acute, mild stressor.

FIG. 15A shows effects of (S)-5-FPT on c-Fos expression in the dorsal hippocampus in wild-type (WT) and Fmr1 knockout (KO) mice. At left is representative 4× magnification image of cresyl violet-stained dorsal hippocampus; at middle are representative 20× magnification images of DAB-stained c-Fos in the CA3 region (of the hippocampus) from WT and KO mice after treatment with vehicle (Veh) or (S)-5-FPT. At right is a plot of c-Fos positive nuclei. (S)-5-FPT did not significantly increase c-Fos expression in CA3 region. FIG. 15B shows effects of (S)-5-FPT on c-Fos expression in the basolateral amygdala anterior (BLAa) in wild-type (WT) and Fmr1 knockout (KO) mice. At left is representative 4× magnification image of cresyl violet stained amygdala; at middle are representative 20× magnification images of DAB-stained c-Fos in BLAa from WT and KO mice after treatment with vehicle (Veh) or (S)-5-FPT. At right is a plot of c-Fos positive nuclei. (S)-5-FPT significantly increased c-Fos expression in the BLAa of KO mice and tended to increase c-Fos expression in the BLAa of WT mice. FIG. 15C shows effects of (S)-5-FPT on c-Fos expression in the inferior colliculus (IC) of juvenile Fmr1 knockout mice exposed for 30 seconds to a 120 dB alarm. (S)-5-FPT, relative to vehicle (Veh), did not affect the number of c-Fos positive cells. At left is a representative image if IC; at center are representative stained images comparing vehicle and (S)-5-FPT; and at right is a plot of c-Fos positive nuclei.

### DETAILED DESCRIPTION

The present technology can replace previous treatments of indiscriminate elevation of 5-HT levels, which have non-therapeutic interactions at multiple 5-HT receptor types. The technology can target specific serotonin receptors, either alone or in combinations, and can replace the ‘shotgun’ approach to modulating serotonin receptors. The technology can minimize or prevent the side-effects caused by indiscriminate elevation of serotonin levels. The present methods, compounds, and compositions can selectively modulate one or more serotonin receptors, also known as 5-hydroxytryptamine receptors or 5-HT receptors. The technology, for example, may modulate one or more of the 5-HT1A, 5-HT1B, 5-HT1D, 5-HT1E, 5-HT1F, 5-HT2A, 5-HT2B, 5-HT2C; 5-HT3; 5-HT4; 5-HTSA; 5-HTSB; 5-HT6; and 5-HT7 receptors. The methods can minimize adverse symptoms without significantly decreasing desirable behaviors (e.g., causing non-therapeutic effects). The methods can show an improvement in behavior in one aspect without causing a decline in behavior in another aspect. The technology provides methods of treating diseases or disorders, along with methods to improve behaviors or symptoms. Symptoms of the diseases or disorders can manifest as psychiatric symptoms, as physical symptoms, or as both. Use and withdrawal from drugs (e.g., SUD) can cause physical and psychiatric symptoms.

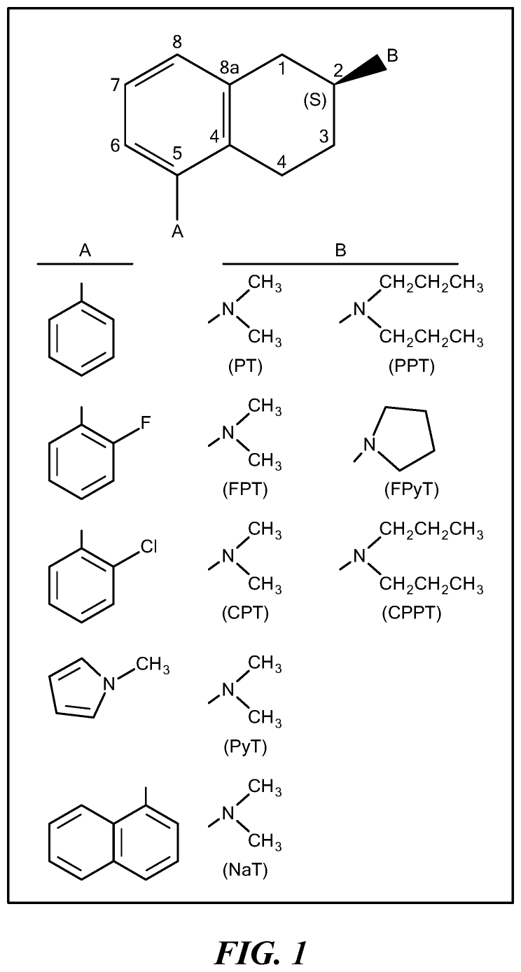

Examples of 5-phenyl-2-aminotetralin (5-PAT) compounds provided by the technology are depicted in FIG. 1. At the top of FIG. 1, the 5-phenyl-2-aminotetralin (5-PAT) moiety is illustrated. The “B” substituent can be illustrated by a general amino formula —NR¹R².

Examples of (S)-5-PAT compounds depicted in FIG. 1 are shown below:

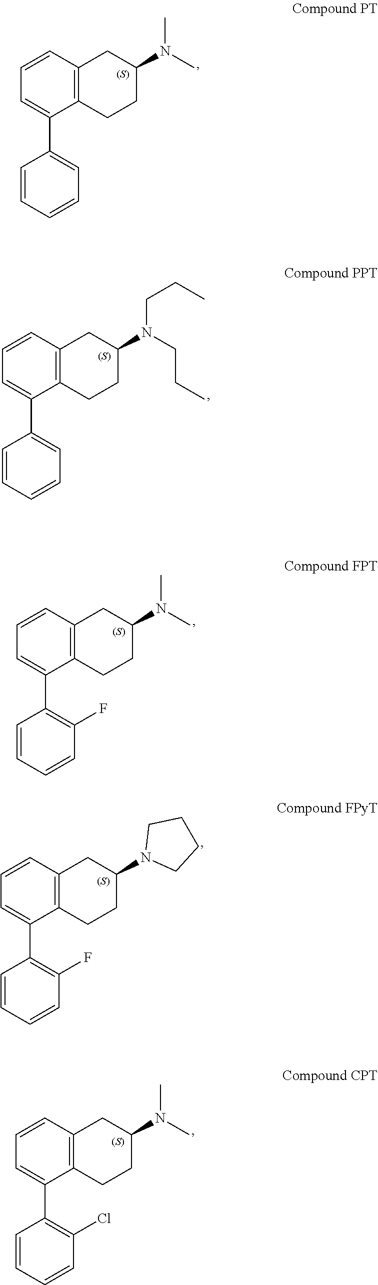

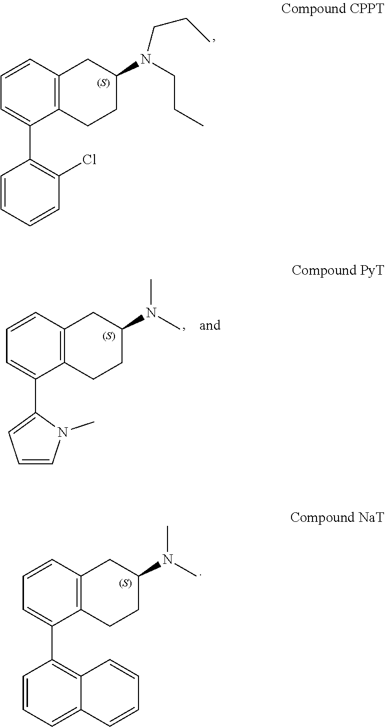

These example structures of FIG. 1 can be represented as a set of Formula (I) below. An aspect of the technology is a method of treating or preventing a neuropsychiatric disease or disorder, that includes administering a therapeutically effective amount of a racemic or enantiomerically pure (preferably at least 70% pure (S)-enantiomer) dual partial agonist at the serotonin 5-HT₇ and 5-HT_{1A} receptors, the dual partial agonist having the structure of Formula (I):

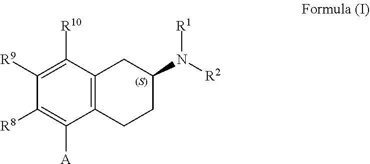

wherein “A” is a ring or ring system, such as a substituted or unsubstituted aromatic or non-aromatic ring or ring system with or without N, O, or S in substitution for one or more carbon atoms, for example, naphthalene, isoquinoline, anthracene,

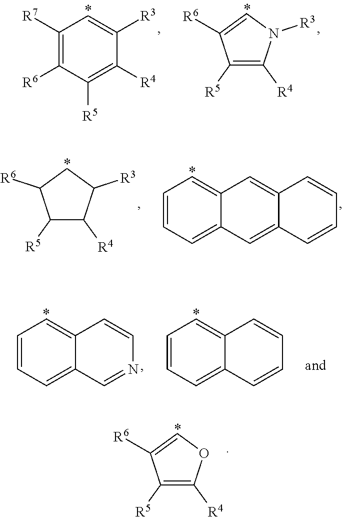

The dual partial agonist can be in the form of a pharmaceutically acceptable salt, hydrate, or solvate thereof.

The dual partial agonist can be wherein each of R¹ and R² is independently hydrogen, substituted or unsubstituted alkyl, alkenyl, or alkynyl; and R¹ and R² may come together to form a substituted or unsubstituted alkyl ring, heterocyclic ring, or aromatic ring;

wherein each of R³, R⁴, R⁵, R⁶, R⁷, R⁸, R⁹, and R¹⁹ is independently selected from hydrogen, halo, hydroxyl, acyl, acyloxy, alkyl, carbonyl, sulfonamide, benzyl, phenyl, naphthyl, heteroalkyl, alkenyl, heteroalkenyl, alkynyl, heteroalkynyl, alkoxycarbonyl, cyano, trifluoroalkyl, trifluoromethyl, trifluoroalkoxy, trifluoromethoxy, nitro, amino, amido, chloro, fluoro, bromo, iodo, aldehyde, haloformyl, carbonate ester, carboalkoxy, alkoxy (attached through 0), methoxy, ethoxy, hydroperoxy, peroxy, ether, carboxamide, primary amine, secondary amine, tertiary amine, primary ketimine, azide, azo diimide, cyanate, isocyanate, nitrate, nitrile, isonitrile, nitrosooxy, nitroso, oxime, 4-pyridyl, 3-pyridyl, 2-pyridyl, carbamate, thiol, sulfhydryl, sulfoxide or sulfinyl, sulfone or sulfonyl, sulfino, sulfo, thiocyanate, isothiocyanate, carbonothioyl, thione, thial, thioic S-acid, borono, boronate, borino, cycloalkyl, and cycloaryl, wherein any two adjacent R groups may optionally come together to form a substituted or unsubstituted carbocyclic, aromatic, or heterocyclic ring or ring system. “A” of Formula (I) can be a 5 or 6 membered substituted or unsubstituted alkyl ring, aromatic or aryl ring, heteroalkyl, heteroaryl, or heteroaromatic ring (wherein one or more carbon atom is replaced by S, O, or N). If A is a heterocyclic ring, A is attached through a carbon atom. As used herein, substitution or substituted can be covalent attachment of any suitable organic group known in the art. For example, A, R¹, R² can be substituted with one or more of the aforementioned substituents independently designated for R³⁻¹⁰.

In an embodiment, the compound of Formula (I) is a compound which is not disclosed in US 2017/0081273A1, which is incorporated herein by reference in its entirety. Also incorporated herein by reference in its entirety is C. K. Perry et al., Synthesis of novel 5-substituted-2-aminotetralin analogs: 5-HT1A and 5-HT7 G protein-coupled receptor affinity, 3D-QSAR and molecular modeling, Bioorganic & Medicinal Chemistry 28:115262 (2020) and Jessica L. Armstrong, Austen B. Casey, Tanishka S. Saraf, Munmun Mukherjee, Raymond G. Booth, and Clinton E. Canal, (S)-5-(2′-Fluorophenyl)-N,N-dimethyl-1,2,3,4-tetrahydro-naphthalen-2-amine, a Serotonin Receptor Modulator, Possesses Anticonvulsant, Prosocial, and Anxiolytic-like Properties in an Fmr1 Knockout Mouse Model of Fragile X Syndrome and Autism Spectrum Disorder, ACS Pharmacology & Translational Science 2020 3 (3), 509-523 DOI: 10.1021/acsptsci.9b00101.

Pharmaceutical compositions of the present technology can include any of the foregoing compounds, or any combination thereof, and one or more pharmaceutically acceptable excipients or carriers.

The dual partial agonist at the serotonin 5-HT₇ and 5-HT_{1A} receptors can be a racemic mixture containing up to 50% pure (S)-enantiomer, or at least 50% pure (S)-enantiomer, at least 60% pure (S)-enantiomer, at least 70% pure (S)-enantiomer, at least 80% pure (S)-enantiomer, at least 90% pure (S)-enantiomer, at least 95% pure (S)-enantiomer, or at least 99% pure (S)-enantiomer, or essentially 100% pure (S)-enantiomer. Chiral synthesis can be utilized, in many cases, to achieve the essentially 100% pure (S)-enantiomer. As used herein, the essentially 100% pure (S)-enantiomer refers to ≥99.0% (wt./wt.) of the (S) enantiomer compared to the combined weight of the (S) and (R) enantiomers, or the essentially 100% pure (S)-enantiomer refers to ≥99.0% area under the curve (AUC) of the (S) enantiomer compared to the combined AUC of the (S) and (R) enantiomers (UV Trace 220/254 nm).

Applications of the technology can involve serotonin receptors distributed in the central nervous system and/or in the periphery. Examples of diseases or disorders that can be treated using compounds, pharmaceutical compositions, and methods of the present technology include SUD, opioid use disorder (OUD), addiction, anxiety, psychoses, depression, autism spectrum disorder, fragile X syndrome, neurological disorders, repetitive behaviors, movement disorders, compulsions, tics, inflammatory bowel disease (IBD), ulcerative colitis (UC), Crohn's disease (CD), gastrointestinal disorders, pain disorders, vasospastic disorders, migraine headache, seizures, epilepsy, social anxiety, addiction withdrawal, drug withdrawal, drug abuse, alcoholism, eating disorders, diarrhea, general inflammation disorders, and miosis. The methods, compounds, and compositions described herein can be used to treat or prevent, in general, a disorder that can be improved by modulation of one or more serotonin receptors.

As used herein but as further described below, not at “physiologically-relevant levels” can refer to a binding constant of 1 μM or greater, 500 nM or greater, or 300 nM or greater. The dual partial agonists, depending on the chemical structure, do not bind one or more of the serotonin 5-HT2A and 5-HT2C receptors at physiologically-relevant levels. For example, the dual partial agonists can bind one or more of the serotonin 5-HT_{2A} and 5-HT2C receptors with a binding affinity of greater than about 300 nM, or greater than about 400 nM, or greater than about 500 nM, or greater than about 750 nM, or greater than about 1 μM, or greater than about 5 μM, or greater than about 10 μM.

For example, the dual partial agonists can bind to one or more of the serotonin 5-HT7 and 5-HT_{1A} receptor at physiologically relevant levels; the dual partial agonists can bind to the serotonin 5-HT7 receptor with a binding affinity (Kᵢ) of less than about 300 nM, or less than about 100 nM, or less than about 50 nM, or less than about 25 nM, or less than about 20 nM, or less than about 10 nM, or less than about 5 nM, or less than about 2 nM, or less than about 1 nM. The dual partial agonists can bind to the serotonin 5-HT_{1A} receptor with a binding affinity (Kᵢ) of less than about 300 nM, or less than about 100 nM, or less than about 50 nM, or less than about 25 nM, or less than about 20 nM, or less than about 10 nM, or less than about 5 nM, or less than about 2 nM, or less than about 1 nM.

Novel 5HT_{1A} and 5HT7 GPCR-targeting compounds are provided which can correct behavioral symptoms of autism, which include repetitive and perseverative behaviors, social deficits, irritability, circadian rhythm disturbances, and anxiety, which are a panoply of symptoms with striking overlap to psychiatric symptoms associated with SUD or drug withdrawal. The examples shown in FIG. 1 are novel, orally-active 5-phenyl-2-aminotetralin (5-PAT)-type compounds that have (stereo)selective high affinity at 5HT_{1A} and 5HT7 GPCRs (G protein-coupled receptors). An example is (2S)-5-FPT, 5-(2′-fluorophenyI)—N,N-dimethyl-1,2,3,4-tetrahydronaphthalen-2-amine, or 5-(2′-fluoro-phenyl)-2-dimethylamino-tetralin), which is a 5HT1A and 5HT7 partial agonist that attenuates repetitive behaviors, promotes social behavior, and does not impact spontaneous locomotor behavior in numerous mouse models involving three distinct wild-type mouse strains. This and other 5-PAT compounds of the present technology can address neuropsychiatric symptoms, and their 5HT_{1A} and 5HT7 affinities and functional activities (full and partial agonism and inverse agonism) can be assessed in a battery of mouse models of psychiatric symptoms associated with withdrawal and abstinence from morphine or THC.

Full-agonist activation of 5HT_{1A} receptors, such as with (R)-8-hydroxy-2-(n-di-propylamino)tetralin (8-OH-DPAT), is untenable because this produces a life-threatening 5-HT syndrome or serotonin syndrome. In general, there appears to be CNS sedative effects followed by CNS stimulation observed clinically with buspirone. However, the present technology has revealed the 5HT7 receptor activity of buspirone (nil, Kᵢ>1.0 μM; see Table 1).

TABLE 1
Affinities (Kᵢ ± SEM, nM) and functions
of select 5-PATs at 5HT1A and 5HT7

| Name | 5HT1A | 5HT7 |
|---|---|---|
| (S)-5-PT | 22 ± 3 | 9.5 ± 1.5 |
| | Partial agonist | Neutral antagonist |
| (S)-5-FPT | 4 ± 0.1 | 5.3 ± 2 |
| | Partial agonist | Partial agonist |
| (S)-5-CPT | 7.5 ± 1.5 | 1.0 ± 0.12 |
| | Partial agonist | Partial agonist |
| (S)-5-PyT | 24 ± 0.2 | 33 ± 4 |
| | Partial agonist | Inverse agonist |
| (S)-5-NaT | 64 ± 12 | 5.2 ± 1.1 |
| | Partial agonist | Partial agonist |
| (S)-5-PPT | 5.9 ± 1.1 | 34 ± 3 |
| | Undetermined | Full agonist |
| (S)-5- | 0.6 ± 0.2 | 5.9 ± 1.0 |
| CPPT | Undetermined | Full agonist |
| (R/S)-5- | 1.3 ± 0.1 | 51 ± 8 |
| FPyT | Undetermined | Undetermined |
| (S)-AS-19 | 240 ± 1.6 | 12 ± 0.9 |
| | Undetermined | Partial agonist |
| Buspirone | 50 ± 5 | >1000 |
| | Partial agonist | Undetermined |

Table 1 shows affinity of several single-enantiomer 5-PAT-type analogs (e.g., FIG. 1) for which chemical synthesis has been completed.

5HT1A and 5HT7 GPCRs are closely linked to SUD withdrawal symptoms. 5HT1A knockout mice show decreased social behavior and enhanced generalized social anxiety, and 5HT_{1A} activation is well-known to produce anxiolytic effects without addiction risk and, also, pro-social effects. The 5HT_{1A} partial agonist buspirone is an anxiolytic that has shown some efficacy to treat opioid withdrawal.

5HT7 knockout causes repetitive and perseverative behaviors, and 5HT7 agonists have been shown to treat neuropsychiatric symptoms in genetic models. Perseveration is an endophenotype of several psychopathologies, and is associated with the opioid system, opioid abuse and withdrawal, and both 5HT1A and 5HT7 receptors contribute to behavioral flexibility. Alterations in inhibitory control might contribute to high relapse risk even after a period of heroin or opioid abstinence. 5HT neurons send projections to the ventral striatum, a foci of addiction and repetitive behaviors, and 5HT1A and 5HT7 receptors are expressed there, and in nearly all circuits linked to neuropsychiatric symptoms associated with drug withdrawal. For example, 5HT7 are expressed in the suprachiasmatic nuclei and are key regulators of circadian rhythms. Co-targeting 5HT1A and 5HT7 receptors is ideal to treat neuropsychiatric symptoms of drug withdrawal.

The presently disclosed 5-PAT compounds have been identified during an effort to discover anti-addiction medications and antipsychotics based on the novel molecular scaffold 4-phenyl-2-aminotetralin (4-PAT), which targets serotonin 5-HT2 GPCRs. Medicinal chemical studies have been broadened to include syntheses of 5-phenyl-2-aminotetralins (5-PATs) (e.g., FIG. 1). 5-PATs can have low to nil activity at 5-HT2 receptors, and the binding affinities are discussed in more detail below. However, compounds related to 5-PATs such as (2S)-5-trimethylpyrazolyl-2-dimethyl-aminotetralin (AS-19) have been reported to have high-affinity, partial agonist activity at 5HT7 receptors. It has also been reported that AS-19 has moderate affinity (unknown function) at 5HT1A. It has since been determined that AS-19 has 20-fold selectivity for 5HT7 over 5HT_{1A} (Table 1):

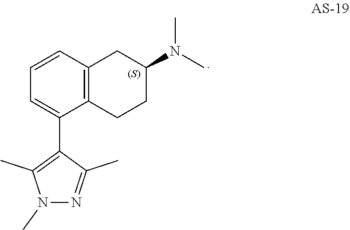

To assess 5HT7 pharmacology of the novel 5-PAT compounds, a monoclonal HEK293 cell line has been generated with high 5HT7 receptor binding density (B_{MAX}=7.7(0.4) pmol/mg). At such a high 5HT7 receptor density, a 5HT7 partial agonist is not expected to appear as a full agonist because receptor reserve is not an issue. Unexpectedly, it is shown herein that certain 5-PATs also have stereoselective high affinity at 5HT1A receptors. Recently, a cell line that can stably-express 5HT_{1A} has been developed, but most data reported herein come from transiently-transfected HEK (affinity) and CHO (function) cells.

For the 5-PAT compounds, the optimal C(2)-amine stereochemistry is the S-configuration for binding at 5HT1A and 5HT7 (FIG. 1, Table 1). Affinity of the corresponding (2R)-enantiomers of 5-PATs in Table 1 are 20-times to 550-times lower than the (2S)-enantiomers. Data for example molecules demonstrates that selectivity for 5HT_{1A} or 5HT7 GPCRs can be achieved. For example, while C(5) phenyl or 2′-halophenyl substituents do not much impact selectivity (5-PAT, 5-FPT, 5-CPT; Table 1), increasing the size of 5-substituents selects for 5HT7 (5-PyT). Altering the C(2)— amine moiety from dimethyl to dipropyl selects for 5HT1A (5HT1A affinity and selectivity of the dipropyl analog 5-CPPT is improved compared to its corresponding dimethyl analog 5-CPT; Table 1). Likewise, altering the C(2)— amine moiety from dimethyl to pyrrolidine (which is a rigid diethyl homolog) selects for 5HT1A (5-FPyT, Table 1). From experiments, substitution at C(6) and C(7) results in low affinity at both receptors, and C(8)-substitution (8-OH-DPAT) provides unacceptable full agonism at 5HT1A. These examples demonstrate the technology can provide selectivity for various 5-HT receptors.

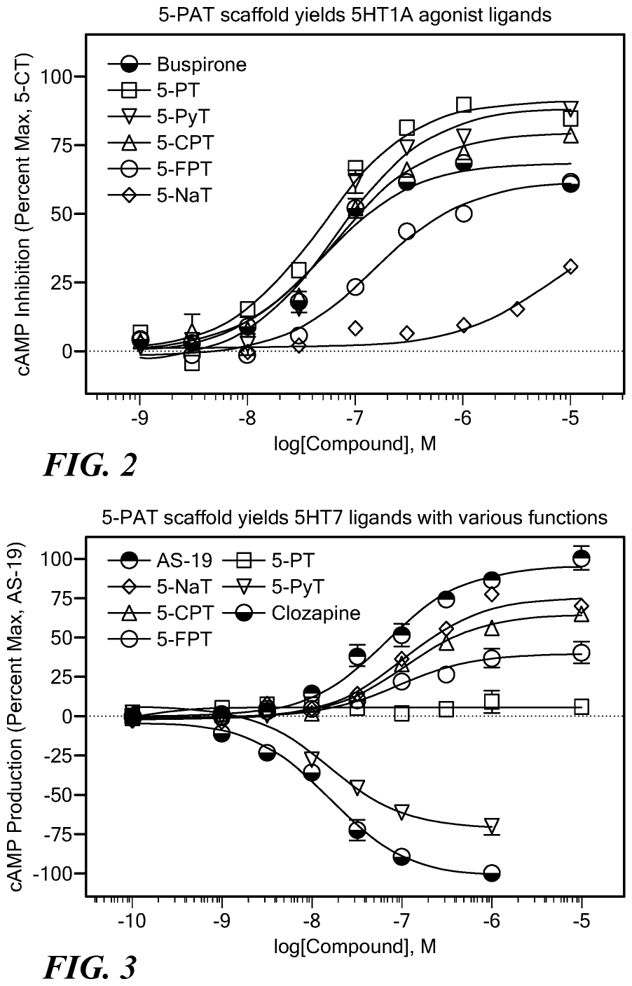

To further describe the 5-PAT Function at 5HT1A and 5HT7 receptors, that is, ability to stabilize agonist vs. inverse agonist conformation(s) of 5HT1A or 5HT7, is more difficult to predict. It is certain that the C(5) substituent impacts function. Thus, the 5-PyT analog with a C(5) methylpyrrole aromatic moiety has about equal affinity at 5HT1A and 5HT7, but, it is an agonist at 5HT1A/Gαᵢ and inverse agonist at 5HT7/Gαₛ regarding cAMP signaling (see FIGS. 2-3 and Table 1). In FIG. 2, the results of measured (S)-5-PAT scaffold yields as 5HT1A agonist ligands are compared with that of Buspirone. In FIG. 3, results of measured (S)-5-PAT scaffold yields with various function as 5HT7 ligands are compared with AS-19 and clozapine. The surprising result of the 5-PyT analog has led to further studies, in particular, initiation of computational chemistry and molecular modeling studies (described below) to study how selected 5-PATs dock at the 5HT_{1A} and 5HT7 binding pockets to impact affinity, selectivity, and function.

Computational chemistry and molecular modeling provide insight on molecular determinants for binding and function of the 5-PATs at 5HT_{1A} and 5HT7. Results indicate medicinal chemical optimization provides opportunities to provide high affinity 5-PAT-type analogs with a range of selectivity at 5HT1A and 5HT7 should focus on C(2)-amine substitution/stereochemistry and C(5)-substitution to optimize steric, electrostatic, and hydrophobic interactions with specific 5HT_{1A} or 5HT7 amino acids to impact affinity, selectivity, and function. The compounds and technology herein focus on the (S)-enantiomer and tailoring of the C(2)-amine substitution and C(5)-substitution.

The receptor specific interaction, has led to studies of affinity and function at other relevant GPCRs for the 5-PAT compounds. 5-PATs are stereoselective high-affinity neutral antagonists at serotonin 5HT_{2B} receptors and moderate affinity agonists at 5HT₂C receptors. At 5HT_{2A} receptors, affinity of (2S)—S-FPT and (2S)-5-CPT is very low (Kᵢ˜900 and 500 nM, respectively). At 5HT_{2B} receptors, however, (2S)—S-FPT has moderate affinity (Kᵢ=60 nM) and (2S)-5-CPT has robust affinity (Kᵢ=11 nM). In functional assays, (2S)—S-FPT and (2S)-5-CPT are devoid of 5HT_{2B} activity up to 100 μM, suggesting neutral antagonism. It is noted that 5HT2B agonist activity is untenable clinically because it can lead to cardiac valvulopathy. 5HT2B antagonists, however, reduce mesoaccumbens dopamine outflow and attenuate amphetamine-induced hyperlocomotion in mice, suggesting 5HT_{2B} antagonism may produce beneficial psychotherapeutic effects. At 5HT2C receptors, (2S)—S-FPT is nearly a full-efficacy agonist with low potency (EC₅₀=230 nM), and (2S)-5-CPT has less potency and efficacy. National Institute of Mental Health Psychoactive Drug Screening Program results summarized below indicate a “clean” profile.

Two novel representative lead 5-PATs, (2S)—S-FPT and (2S)-5-CPT, are high-affinity 5HT1A/5HT7 partial agonists. The compounds have very low affinity at 5HT2A receptors, clinically beneficial antagonist activity at 5HT_{2B} receptors, and are low potency 5HT2C agonists (indicates use for alleviating cognitive dysfunction), without activity at 50 CNS/peripheral sites.

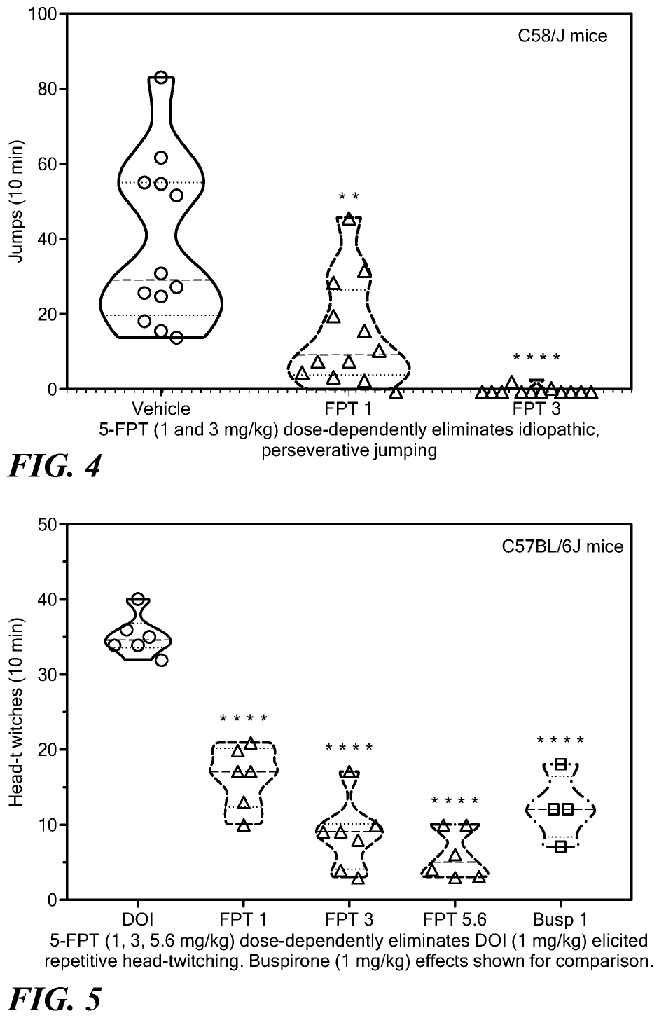

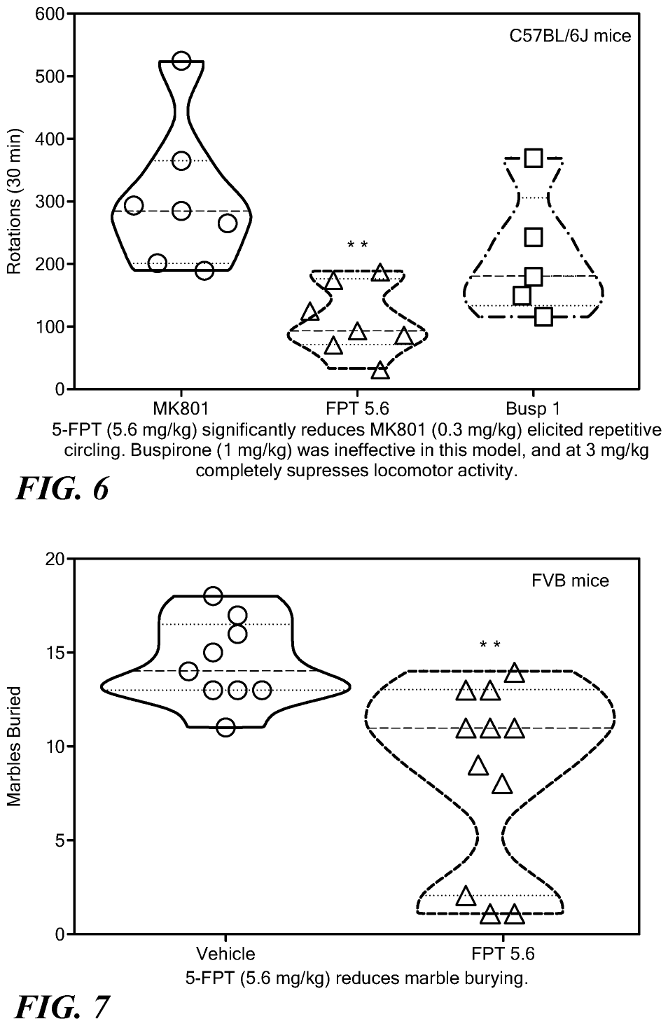

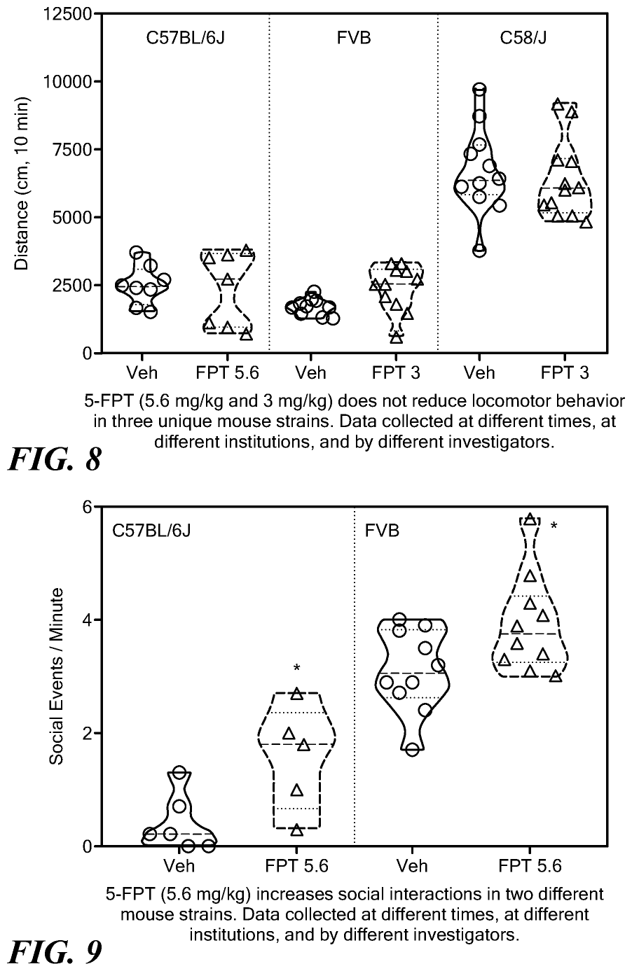

(2S)-5-FPT eliminates repetitive and perseverative behaviors, without affecting general locomotion, suggesting anxiolytic effects. (2S)-5-FPT has been tested in four, heterogeneous models of repetitive and perseverative behavior that includes three unique mouse strains: 1) idiopathic repetitive jumping in 058/J mice (FIG. 4); 2) (±)-DOI (5HT2 agonist) elicited head-twitching in C57BL/6J mice (FIG. 5); 3) MK801 (NMDA antagonist) elicited circling in C57BL/6J mice (FIG. 6); 5) marble burying in FVB mice, an anxious and hyperactive WT strain (FIG. 7). (2S)-5-FPT reduces or eliminates repetitive behavior in all models, without altering locomotor behavior on its own (FIG. 8). In contrast, buspirone, which has nil 5HT7 affinity (Table 1), reduces DOI-elicited repetitive behavior, but also reduces locomotor behavior (data not shown). In fact, 3 mg/kg buspirone, by itself, causes stupor-like catatonia, likely due to its D2 receptor activity.

(2S)-5-FPT increases social interactions, and does not cause serotonin syndrome, suggesting pro-social effects without toxicity (FIG. 9 and Table 2). (2S)-5-FPT significantly increases the number of initiated social interactions between littermates in two unique mouse strains. In FIG. 9 and in Table 2, it is shown that even at the highest behaviorally-effective dose (5.6 mg/kg), (2S)-5-FPT does not produce serotonin syndrome-like symptoms, including flat body, forepaw treading, moon walking, piloerection, Straub tail, or tremor, but it decreases rearing, suggesting in vivo 5HT_{1A} activation.

(2S)-5-FPT is neurobehaviorally active after oral administration. (2S)-5-FPT is orally active and readily crosses the blood-brain barrier. For example, (2S)-5-FPT readily crosses the blood-brain barrier, with μg levels achieved in mouse brain at 30, 60, and 90 minutes after systemic administration, quantified by liquid chromatography-mass spectrometry/mass spectrometry. Levels were lower in plasma relative to brain, indicating (2S)-5-FPT is rapidly cleared in the periphery.

TABLE 2
(2S)-5-FPT (5.6 mg/kg) does not cause serotonin syndrome-like behavior in BL/6J mice.

| | Flat | Forepaw | Head | | Moon | Pilo- | | Straub | |
|---|---|---|---|---|---|---|---|---|---|
| Treatment | Body | Tread | Weave | HTR (n) | Walk | erection | Rears (n) | Tail | Tremor |
| Vehicle | 0(0) | 0(0) | 0(0) | 1.1(0.5) | 0.1(0.1) | 0(0) | 27(5) | 0(0) | 0(0) |
| 5-FPT | 0(0) | 0(0) | 0(0) | 0(0) | 0.3(0.2) | 0(0) | 9(3) | 0(0) | 0(0) |

Unlike benzodiazepine anxiolytics which can contribute significantly to opioid overdoses, compounds that activate 5HT_{1A} and/or 5HT7 receptors are unlikely to be addictive, and the 5-PATs have no functional activity at DAT, hERG, 5HT2B (ADM ET data is discussed below). The 5-PAT chemotype is amenable to molecular diversity, and analogs proposed exploit new chemical space regarding 5HT1A and 5HT7 receptor molecular interactions that translate to agonist function. The 5-PAT scaffold has a chiral center at the C(2) amine position (FIG. 1), enhancing 3D molecular diversity that impacts pharmacology, i.e., the 3D arrangement of 5-PAT chemical moieties is a key molecular determinant for 5HT1A and 5HT7 receptor affinity and function. Moreover, the 2-aminotetralin scaffold is relatively rigid, and rigid ligands often have higher receptor affinity because entropic factors are less relevant. Also, rigid ligands can be utilized as templates to define 3D arrangement of residues involved in ligand binding and stabilization of agonist receptor conformation(s). Rigid chemical scaffolds often have less complex metabolism, avoiding potentially toxic species. Thus, the 5-PAT scaffold is superior regarding high affinity at target, lack of affinity at off-targets, and information learned about target GPCR structure. In addition, the 5-PAT scaffold is relatively small (MW=270 for 5-FPT), with favorable lipophilicity (cLogP=4.5) for oral and brain penetration. Efficient Suzuki-coupling chemistry and chiral stationary-phase HPLC enables efficient preparation of single enantiomers in sufficient quantity (˜200 mg) for in vitro pharmacology and in vivo behavioral studies. Single enantiomer synthesis can be achieved using commercially-available 2R- or 2S-5-methoxy-2-aminotetralin starting material. 5-PATs are convertible to hydrochloride salts for high water solubility and oral activity.

To acquire ADMET data, representative PATs have been submitted to the NIMH Psychoactive Drug Screening Program and the NIDA Chemistry and Pharmaceutics Branch to delineate absorption, distribution, metabolism, and toxicity (ADMET) parameters. Evaluation at 50 CNS/peripheral sites indicate very low (Kᵢ>1 μM) or nil (Kᵢ>5 μM) affinity for adenylyl cyclase, adenosine, adrenergic, benzodiazepine, cholinergic, dopamine, GABA_{A}, hERG, histamine, opiate, NMDA, PCP, PLC, sigma, DAT, NET, SERT, ion channels (Ca⁺⁺, Cl⁻, K⁺). Functional studies indicate hERG activity is nil (IC₅₀>2.0 μM), thus, no cardiotoxicity is expected; in-lab results document no activation of 5HT_{2B} GPCRs (no cardiotoxicity). DAT functional activity also was nil. The inhibitory effects on CYP activity in human liver microsomes has been measured for CYP1A2, CYP2B6, CYP2C9, CYP2C19, CYP2D6, and CYP3A4; no inhibition has been observed at 10 μM. Also, no toxicity has been observed to human hepatocyte Fa2N-4 cells. Incubation at 10 and 100 μM with rat, rabbit, dog, monkey, and human liver microsomes indicated predictable N,N-dealkylated and aromatic hydroxylated metabolites.

In addition to their pharmacotherapeutic potential in SUD, the compounds herein also provide new SAR information useful for drug discovery targeting 5HT_{1A} and 5HT7 GPCRs. In this regard, the comparatively rigid 5-PAT molecular scaffold is an ideal probe to pair with molecular modeling studies for delineation of 3D molecular determinants for 5HT1A and 5HT7 binding and function to aid in understanding structure and function of 5HT1A, 5HT7. Notably, there is not an available crystal structure for either 5HT_{1A} or 5HT7.

### 5HT1A and 5HT7 Structure-Activity Relationship (SAR)

There is close (˜47%) sequence homology between 5HT1A and 5HT7 GPCRs in the transmembrane domains (TMDs), where orthosteric ligands bind, and, most reported ligands bind to both receptors. A notable exception is buspirone. It is believed that it is first reported (see Table 1) herein that buspirone has nil affinity Kᵢ>1 μM) at human 5HT7 receptors. Buspirone is the only approved drug that has predominant 5HT1A(partial) agonism. It is a relatively large (MW=385), highly flexible molecule, and such structures have high entropy and myriads of conformational possibilities, which are not useful for inferring GPCR 3D structure, and with potentially numerous off-targets and complex metabolism profiles. In fact, buspirone binds with various functional activities at several 5HT receptors and dopamine D2, and, in humans, buspirone is converted to 8 different oxidized and hydroxylated metabolites with unknown pharmacology and toxicology. (R)-8-hydroxy-2-(n-di-propylamino)tetralin (8-OH-DPAT), related to the 5-PT chemotype, is a high-affinity full-efficacy 5HT1A agonist, long-known for its propensity to cause serotonin syndrome, and, it is a low-potency 5HT7 partial agonist. Another full-efficacy 5HT1A agonist, repinotan, which is a bi-valent chroman/thiazole linked by a flexible 4-carbon chain, caused nausea and vomiting in clinical trials. In general, the literature teaches that large flexible chemotypes and full activation of 5HT1A should be avoided.

There are no approved 5HT7-selective drugs and only 4 commercial agonist probes. There is available AS-19, a partial agonist structurally related to (2S)-5-PATs, and selective for 5HT7 over 5HT1A(12 nM vs. 240 nM, see Table 1), suggesting 5HT1Avs. 5HT7 selectivity can be achieved using the 5-PAT scaffold. AS-19 is an oil and has not been developed as a drug. There are also 5HT7 agonists reported that have a tetrahydronaphthalene core substituted with 1-piperazine and linked to a large (MW-450) highly flexible 5-carbon alkyl chain ending in aryl and biaryl systems, i.e., LP-211 and related analogs. Human receptor data is reported only for LP-211, showing it to be only about 25X selective for 5HT7 over 5HT1A receptors. Moreover, the case for LP-211 being an agonist is not compelling given the same group that reported LP-211 is an agonist also reported that LP-211 is an insurmountable antagonist of the classical 5-HT7 agonist 5-carboxamidotryptamine (5-CT) in human embryonic kidney (HEK293) cells. In fact, recently, LP-211 was identified as a high-affinity long-acting inhibitor of human 5-HT7 receptor binding and function in cell lines. Unfortunately, too, large highly flexible compounds such as the LP analogs, are expected to have many off-targets, complex metabolism, and cannot be used to infer GPCR structure-function information. Moreover, the 5-carbon alkyl chain and other lipophilic moieties of the LP compounds make them too lipophilic (cLogP=6.1), i.e., LogP values >5 have great potential for toxicity. There is also a highly-flexible pyrazol-substituted-phenethylamine compound (E-55888) reported as a ‘selective’ 5HT7 full agonist, but, this molecule also has high entropy with numerous conformational possibilities due to an un-tethered phenethylamine moiety. Encouragingly, data for the 5-PAT molecules reported (FIG. 1, Table 1) gives confidence surrounding selectivity for 5HT1A or 5HT7 GPCRs. Further knowledge is presented herein describing molecular determinants for agonist vs. inverse agonist activity.

Advantages of the 5-PAT Chemotype for Translation of 5HT1A and 5HT7 Agonists includes several aspects. As discussed above, in contrast to known 5HT1A and/or 5HT7 ligands, the 5-PAT scaffold is comparatively small (MW=270 for 5-FPT) and rigid, with favorable lipophilicity (cLogP=4.5) for oral and brain penetration; as indicated LogP values >5 have great potential for toxicity. Rigid ligands often have higher receptor affinity because entropic factors are less relevant. Also, rigid ligands can be utilized as templates to define 3D arrangement of residues involved in ligand binding and stabilization of agonist receptor conformation(s). Moreover, such compounds have less complex metabolism, avoiding potentially toxic species. Metabolism of PATs has been determined in 5 species, and there is predictable N,N-dealkylation and aromatic hydroxylation to 3 polar metabolites, which are easily excreted. Thus, there is confidence that the chosen medicinal chemical scaffold is endowed with a 5HT1A and 5HT7 pharmacophore (selectivity dependent on structural modification), oral activity, brain penetration, limited and predictable metabolism, and with a suitable safety/side effect profile for translational studies. The 5-PATs proposed herein provide insight into 5HT1A and 5HT7 GPCR structure/function, and, mechanistically link 5HT1A and 5HT7 to beneficial effects of 5-PATs to treat neurobehavioral withdrawal symptoms associated with morphine and THC.

### Molecular Pharmacology Strategy and Methods

Assays measuring ligand affinity and function at GPCRs expressed in clonal cells, including, point-mutated receptors to validate molecular modeling results and iteratively inform synthesis studies have been described in recent papers from this work (methods are QuikChange II, Agilent). Affinity has been assessed at human 5HT1A, 5HT7 and off-targets, including 5HT2A, 5HT2B, 5HT2C, D1, D2, and α1A, M3, M4, ρ-opioid, CB1, and CB2 GPCRs (DNA cloned in pcDNA3.1+vector, cdna.org), as well as at the corresponding C57BL/6 mouse GPCRs (pCMV6-Entry vector, Origene). Recently isolated monoclonal HEK293 cells (ATCC, CRL-1573) that stably express 5HT7 receptors (B_{MAX}=7.7 pmol/mg protein determined by [³H]5-CT saturation binding, have been used for all preliminary data above) and also monoclonal CHOK1 cells (ATCC, CCL-61) stably expressing 5HT1A receptors (B_{MAX}=0.9 pmol/mg protein determined by [³H]5-CT saturation binding). HEK293 cells have endogenous Gαₛ-coupled 5HT GPCRs that confound measurement of 5HT1A function via Gαᵢ. The Lance Ultra cAMP kit (Perkin-Elmer) is used to measure GPCR Gαₛ and Gα_{q} cAMP signaling, and the IP One HTRF kit (Cisbio) is used to measure GPCR Gα_{q} signaling.

For data analysis, all in vitro pharmacology experiments are performed a minimum of three times. Nonlinear regression, curve-fitting algorithms using GraphPad Prism 8 can be used for determination of Kᵢ, EC₅₀, Emu(values of 5-PTs. For promotion of 5-PAT 5HT1A and 5HT7 Agonists to In Vivo Studies: Pharmacology Go/No Go, evaluation steps are outlined below.
A. All new candidate ligands assessed for affinity at 5HT1A/5HT7; Progress to B.
B. 5HT1A and/or 5HT7 affinity 50 nM: tested for receptor function; Progress to C.
C. If agonist EC₅₀ potency at either 5HT1A and/or 5HT7≤100 nM, analog is assessed for affinity and function at off-targets noted above. Compounds must then meet one of the following criteria to advance to in vivo studies: 1. For dual 5HT7/5HT_{1A} agonists, 10-fold selectivity (measured by Kᵢ) over off-targets 2. For 5HT7 selective agonists, 15-fold selectivity over 5HT1A and off-targets; C. For 5HT1A selective agonists, Th-fold selectivity over 5HT7 and off-targets. The more stringent selectivity criterion for 5HT7-selective and for 5HT1A-selective agonists is to clearly differentiate 5HT7 and 5HT1A contributions to their psychopharmacology. In the event that human and mouse receptor affinities diverge, mouse receptor affinities can be prioritized for advancement to behavioral assays, e.g., a highly selective human 5HT1A agonist that is not selective at mouse 5HT1A will not inform behavioral mechanisms. It is contemplated that three analogs will advance to Aim 3: one 5HT1A-, one 5HT7-, and one dual 5HT1A/5HT7-selective agonist.

A model to assess in vivo efficacy of 5-PATs to reverse neurobehavioral symptoms elicited by morphine withdrawal in adult male and female C57BLJ6J mice is as follows. Efficacy to correct repetitive behaviors, perseverative behavior, social behavior, acute stress coping or depression-like activity, and circadian activity disturbances is measured. Effects of 5-PATs to buspirone (Buspar) are compared. Buspar is an approved 5HT1A agonist anxiolytic that has efficacy in clinical studies to treat opioid withdrawal symptoms and reduce repetitive behaviors, and in preclinical studies to enhance social interactions, and to attenuate behaviors modeling addiction. There are no approved 5HT7 agonists to which comparisons can be made, and many of the compounds herein have higher affinity at 5HT7 than the most commonly used, commercially-available 5HT7 agonist, AS-19 (Table 1).

A proposed model, Table 3 (behavioral assessment below), provides schematic descriptions of vivo testing. For behavioral assessments, each subject can be observed in two out of four (total) assays in a single day, with timed intervals between assays as noted. It is envisioned that all behavioral observations will be completed within 80 minutes of treatment administration; all PATs tested, to date, maintain psychotherapeutic efficacy in preclinical models for at least two hours after systemic administration. The grouping of behavioral observations can stay consistent for all treatment groups: Assessment of behaviors is in an open-field, focusing on repetitive behaviors, followed by social behaviors in one group of animals (Group A), and perseverative behavior and working memory in the Y-maze, followed by testing in a tail suspension assay in a separate group of animals (Group B) (Tables 3, 4-5). These split groupings have been designed to ensure the parent compound remains active throughout behavioral testing and to prevent confounds from a potential active metabolite(s), e.g., N-dealkylated and aromatic-hydroxylated compounds identified in blood samples by LCMS. Related, Group A involves a single testing environment, so it was naturally chosen to group the tasks in Group A, delegating the other two tasks to Group B. A separate group of animals can be monitored for circadian rhythms for seven consecutive days of natural withdrawal.

TABLE 3
Behavioral Assessment: Morphine Precipitated
and Natural Withdrawal

Vehicle or morphine at 8:00 AM and 6:00 PM daily for 6 days
Last vehicle or morphine injection administered on day 7 at 8:00 AM
-120 min. interval-**
Treatment: vehicle, naloxone, 5-PT + naloxone, or buspirone + naloxone
-20 min. interval-
Testing: In 1. and 2. (Group A) or 3. and 4. (Group B)
1. Repetitive and locomotor behavior assessed for 20 min.
-20 minute interval-
2. Social interactions assessed for 20 min.
-10 minute interval-
Euthanasia, brains prepared for c-Fos immunohistochemistry
3. Performance in Y-maze assessed for 20 min.
-35 minute interval-
4. Performance in tail suspension assay assessed for 5 min.
-10 minute interval-
Fos Analyses: Euthanasia, brains prepared for immunohistochemistry
**Behavioral Assessment for Morphine Natural Withdrawal: interval between last vehicle or morphine injection and treatment is 7 - days.

Regarding general procedures for inducing morphine withdrawal, mice (N=10 female and 10 male/group) can be treated with escalating doses of morphine (20-100 mg/kg) for 7-consecutive days, a regimen known to produce tolerance and alterations in behavior during precipitated or protracted withdrawal. Mice can be injected subcutaneously (SC) with morphine (or vehicle) twice daily (8 AM, 6 PM): 20 mg/kg on day 1 and 2, dose increased by mg/kg on successive days, to 100 mg/kg on days 6 and 7 (only one injection on day 7 at 8 AM). One morphine group can undergo naloxone (30 mg/kg)-induced withdrawal 2-h after the last morphine injection on day 7. A separate morphine group can undergo natural withdrawal for 7-days.

To measure reversal of behavioral effects of morphine withdrawal, efficacy of 5-PATs to alleviate neurobehavioral abnormalities caused by morphine withdrawal can be determined by administering a 5-PAT (1, 3, 10 mg/kg, SC) 10 minutes before naloxone-induced withdrawal or on the 8th day of natural withdrawal. 20 minutes after 5-PAT treatment, behavioral screening begins. Efficacy can be compared to control groups: 1) mice treated with vehicle only; 2) mice treated with morphine+vehicle; 3) mice treated with morphine plus buspirone (0.1, 1, 3 mg/kg), natural withdrawal; 4) mice treated with morphine plus buspirone plus naloxone, precipitated withdrawal. An open field chamber (43×43×31 cm) is used for assessments of repetitive behaviors, locomotor activity, and social interactions. A Y-Maze (each arm 60×10×30 cm) is used for spontaneous alternation and perseverative behavior testing. An overhead CCD camera linked to a PC running Ethovision XT software (Noldus) is used to record behaviors. After sessions are completed, investigators blind to treatment watch videos and score behaviors not automatically detected by Ethovision. A hook and wire is used for the tail suspension assay. All compounds are dissolved in MilliQ water (except THC), sterile-filtered, and administered sc at 0.01 ml/g body weight.

To assess opioid withdrawal-elicited repetitive behaviors, Group A: Twenty (20) minutes after treatment (vehicle, 5-PAT, or buspirone), each mouse is placed in the center of the open-field, and Ethovision, equipped with the Mouse Behavior Recognition Module, records frequency of full rotations, grooming, jumping, rearing, total distance, velocity, distance moved without stopping (distance/bout), thigmotaxia, and time immobile. Scoring is done by hand while replaying video recordings, repetitive behaviors, including, non-ambulatory horizontal movements (weaving), paw twitches, and head twitches. Also, careful analysis of self-grooming is done, recording syntactic chains (repeated, sequential stereotyped movements) and non-chain grooming, as described in detail, and will cases of diarrhea are documented. Behavior is scored for 20 minutes. Mice are then returned to their home cages for a 20 minutes interval, after which social behaviors are assessed.

To measure social behavior during opioid withdrawal, Group A: One control mouse of the same sex and one mouse treated with 5-PAT, buspirone, or vehicle (morphine group) are placed in the center of the open field, and freely explore for 20 minutes The number of self-initiated social interactions (self-directed approach from one mouse to the other, resulting in physical contact) and the number of self-grooming bouts is calculated by Ethovision software. Social interactions that result in aggressive behavior are also scored by investigators.

To record opioid withdrawal-elicited perseverative behavior, Group B: Mice are tested in the hippocampus-dependent, Y-maze spontaneous alternation task that takes advantage of the instinct to explore novel environments. With this task, determination of the effects of opioid withdrawal on perseverative behavior and spatial working memory, and their modulation by 5-PATs are done. Methods are based on previously reported experiments. Twenty (20) minutes after treatment with a 5-PAT, buspirone, or vehicle, each mouse is placed into the center of a three-arm Y-maze. Arm entries are recorded for 20 minutes by Ethovision, and alternation score will be calculated: [total alternations (entry into three different arms consecutively)/(total arms entered—2)] *100.

To describe the tail suspension assay, Group B: here is determined efficacy of 5-PATs to decrease immobility time in a tail suspension assay, a model of depression-like activity that has predictive validity for antidepressant activity and advantages over other preclinical models of depression. 35 minutes after performing in the Y-maze, mice are suspended by their tails to hooks connected to perpendicular wires (to prevent tail climbing). Visual observation of each mouse is conducted, and record immobility time over 5 minutes.

5-PAT Modulation of central morphine elicited C-Fos Expression has been described in the literature. Literature reports provide proof-of-concept that central c-Fos expression during drug withdrawal is a moveable biomarker with predictive validity for drug development to treat drug withdrawal symptoms. Compelling evidence shows that drug withdrawal and psychiatric symptoms (e.g. anxiety) are associated with hyper-excitability of overlapping neural circuits. Numerous studies across species from rodents to swine to humans report alterations in Fos (c-Fos, FosB) expression in numerous neural systems associated with withdrawal from drugs of abuse including opioids and cannabinoids, providing evidence that Fos expression is a biomarker of neural activity associated with withdrawal. Importantly, many of these neural circuits, e.g. hypothalamus, amygdala, and bed nucleus of the stria terminalis (BNST), are implicated in psychiatric symptoms of withdrawal, and c-Fos expression, for example in the amygdala, correlates with the development of some drug-seeking behaviors. Recent clinical studies report that acute treatment with a serotonin receptor modulator with 5HT1A agonist properties, i.e. psilocybin, can reduce anxiety and change engrained behaviors, providing precedence that a single administration of a 5HT1A and/or 5HT7 receptor modulator might reverse neural circuit perturbations caused by chronic morphine (or THC) administration. Importantly, there is also precedent that acute treatment with pharmacological agents can reverse c-Fos expression changes induced by opioid withdrawal, and at least one, the α2 agonist, clonidine, has shown reliable efficacy to treat opioid withdrawal in humans. Moreover, the α2 agonist, lofexidine (Lucemyra), was recently approved by the FDA to treat physical symptoms of opioid withdrawal.

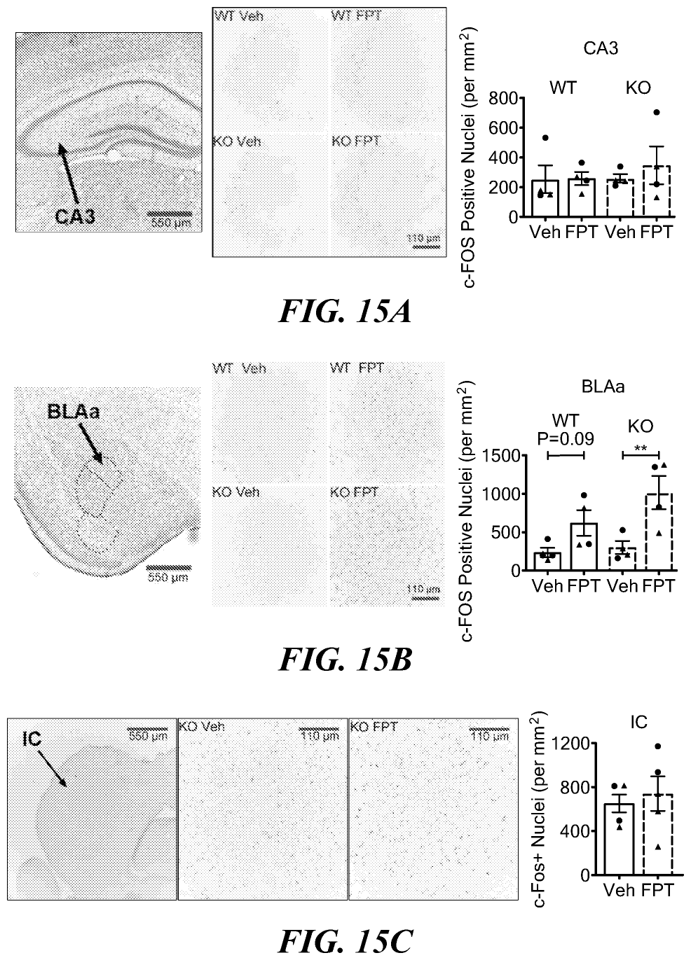

To measure c-Fos immunohistochemistry (IHC), 90 minutes after receiving intervention treatment with the highest dose of a 5-PAT, buspirone, or vehicle, and 10 minutes after the last behavioral assessment, mice are deeply anaesthetized with isoflurane inhalation (up to 5%), and then transcardially perfused with ice-cold tris buffered saline (TBS), followed by 4% paraformaldehyde in TBS. Brains are excised and post-fixed overnight in 4% paraformaldehyde, followed by 20% glycerol in TBS for at least 24 hrs. Coronal sections (40 μm) traversing regions of interest then be collected using a Leica CM1950 cryostat, and placed in cold TBS. c-Fos IHC is performed using free-floating IHC methods as have been previously described, but with a new primary antibody. A protocol with the Abcam [2H2] (ab208942) mouse monoclonal antibody has been recently optimized, which has essentially no cross-reactivity. An HRP-conjugated secondary antibody is used for visualization by the biotin-streptavidin technique using 3,3′-diaminobenzidine as the chromagen (Vectastain Elite, Vector Laboratories). Representative data with this technique are shown in FIG. 14 and FIGS. 15A-15C. Examination of c-Fos expression in frontal cortex, hypothalamus, nucleus accumbens, ventral tegmental area, amygdala, BNST, and hippocampus are conducted.

Circadian rhythms during opioid withdrawal can also be monitored. Numerous reports show that the 5HT7 receptor regulates circadian rhythms. Here, a separate group of mice and one 5HT₇-selective 5-PAT are used to test its effectiveness at reversing circadian disturbances induced by opioid withdrawal. After 7 days of twice daily morphine treatment, mice are implanted subcutaneously with a telemeter. Upon recovery, mice are returned to clean home cages, then housed in isolation cabinets for assessment over the next 7 days. Mice are treated daily, at 8:00 AM—one hour after lights on in a 12 hr light/dark cycle—with vehicle or a lead 5HT₇-selective 5-PAT (3 mg/kg or most effective dose in behavioral assays). Continuous monitoring is done, and analysis of activity, heart rate, and body temperature using the E-Mitter telemetry system (Starr Life Sciences).

Regarding THC and 5-PATs efficacy to reverse effects of THC withdrawal, experiments can be as follows: Repeated administration of THC followed by CB1 inverse agonist, rimonabant, treatment elicits withdrawal symptoms in mice with similarities to those observed during opioid withdrawal. Beginning with 5-FPT and then extending to new analogs, efficacy of 5-PATs to alleviate neurobehavioral symptoms elicited by THC withdrawal in mice is assessed. Results are compared to control groups. Testing procedures have the following exceptions: 1) THC is dissolved in MilliQ water with propylene glycol, dimethyl sulfoxide and Tween-80; 2) Mice are treated twice daily with 50 mg/kg THC′ for seven consecutive days; 3) Rimonabant, 3 mg/kg, is used to induce withdrawal. Natural withdrawal is elicited. 5-PAT modulation of behaviors, c-Fos expression, and circadian activity impacted by THC withdrawal is performed.

The experiments described herein involve data analysis protocols. Transparent data reporting to optimize the predictive translational value of preclinical research, and adherence to ARRIVE (Animal Research: Reporting of In Vivo Experiments) guidelines is followed, i.e., randomization procedures, blind testing, and subject numbers with sufficient power to observe statistically meaningful and replicable differences between treatment(s). Experiments in this technology assess drug treatment, dose, and sex effects, which can be statistically analyzed by ANOVAs with multiple comparisons. Clearly articulated goals of the project are stated, to avoid p-hacking or “fishing” for interactions in ANOVA results. Number of animals is based on calculation of effect size using 5-FPT in mice, and power analysis, with estimate of variance obtained from data published from these studies, and from literature reports of the mouse phenotypes. Sample size analysis indicated groups of 10 animals of each sex are sufficient to provide a minimum power of 0.8 with alpha set at 0.01. This statistical plan is appropriate for the experimental methodology, and supports relevance of the preclinical research outcomes, i.e., to assess novel 5HT7/5HT_{1A} agonists for efficacy in reversing neurobehavioral symptoms of opioid and cannabinoid withdrawal. Statistical analyses is performed using GraphPad Prism 8 and StatMate software is used for power analyses. Questions of a statistical nature can be answered using the literature and consultation with the Northeastern Bouvé College of Health Sciences Biostatistics Service Center Data Analysis for c-Fos, IHC: Quantitative measurement by individuals blinded to treatment conditions is performed using NIH Image J software as is previously described. One-way ANOVA with Tukey's post-hoc tests will be used to examine treatment effects on c-Fos positive puncta. #

As discussed above, single enantiomer synthesis can be achieved using commercially-available 2R- or 2S-5-methoxy-2-aminotetralin starting material. For chemical synthesis, Scheme 1 below indicates optimized methods to obtain single enantiomers of 5-FPT and other analogs shown in Table 1 and FIG. 1 in 6-steps. In Scheme 1, 5-Br-tetralone (1), obtained by reacting 1-tetralone with bromine/AIC13, is reduced to give the corresponding alcohol (2) that is treated with pTSA to obtain the olefin (3). m-Chloroperbenzoic acid (mcpba) is reacted with the olefin (3) to obtain the epoxide (4), that is treated with pTSA to obtain key intermediate 5-Br-2-tetralone (5). 5-Br-2-tetralone (5) can be reacted with a wide variety of available boronic acid derivatives (6) for use in the Suzuki-Miyaura cross coupling reaction to efficiently obtain derivative with variations at the 5-position. In the last step of Scheme 1, reductive amination can introduce a various mono- and di-substituted alkyl, aryl, cycloalkyl and cycloaryl amine moieties at the 2-position. Thus, Scheme 1 has given 5-FPT racemate (8), resolved by polysaccharide-based chiral stationary phase-HPLC, (CSP)-HPLC, to obtain (+)-(2R)- and (−)-(2S)-5-FPT.

Stereospecific synthesis is described in Scheme 2. To determine the absolute stereochemistry at the 2-position, (2S)-5-FPT as well as other analogs in FIG. 1 and Table 1 have been synthesized from commercially available (2S)-5-methoxy-2-aminotetralin. Triflation of the 5-phenol group has been unsuccessful using triflicanhydride whereas use of N-phenyltriflimide has been successful. Unfortunately, the starting material is expensive, thus, chiral stationary phase HPLC (CSP-HPLC) can be the default to obtain single enantiomers. Alternative synthetic strategies have been demonstrated in previous papers on 5-(substituted)-2-dimethylaminotetralins. Chart 1 presents synthetic building blocks for 5-PATs.

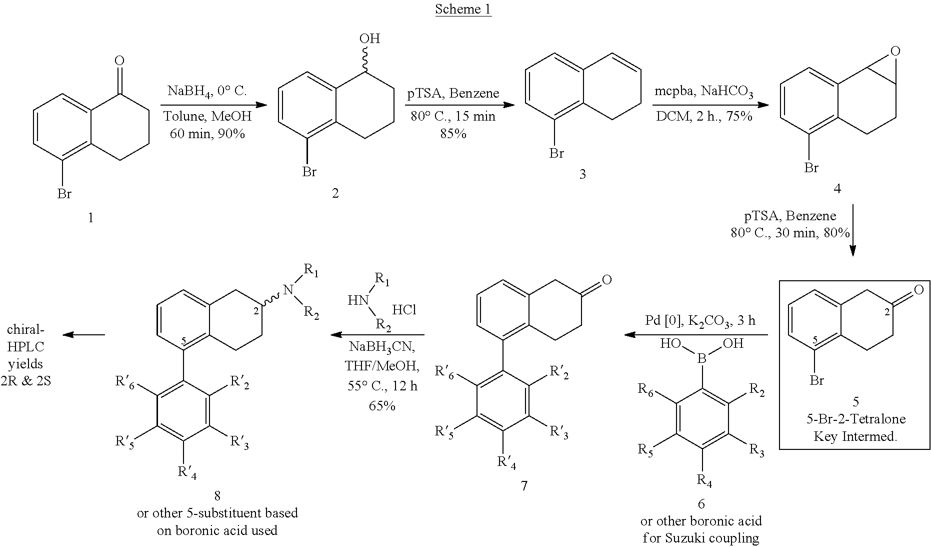

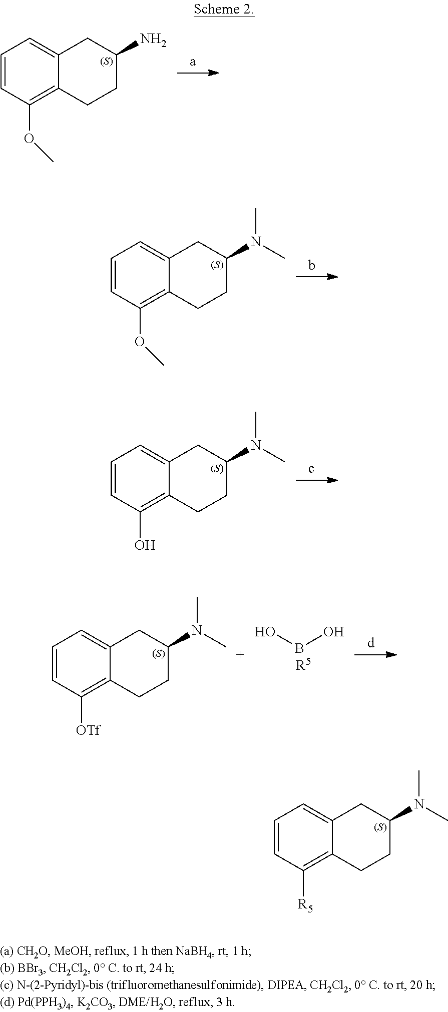

CHART 1
Synthetic building blocks for 5-PATs.
Hydrogen & Halogen Binding Groups Hydrophobic /Aromatic

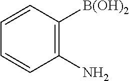

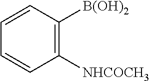

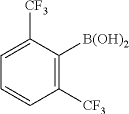 [SMILES: OB(O)c1c(C(F)(F)F)cccc1C(F)(F)F]

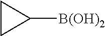

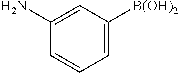

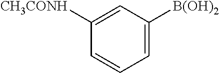

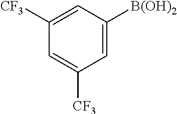 [SMILES: OB(O)c1cc(C(F)(F)F)cc(C(F)(F)F)c1]

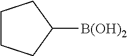

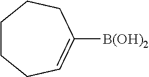

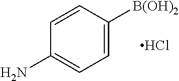

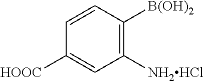

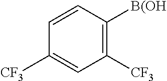

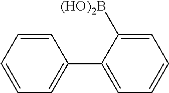 [SMILES: OB(O)c1ccccc1-c1ccccc1]

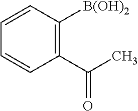

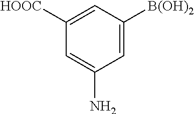 [SMILES: Nc1cc(B(O)O)cc(C(=O)O)c1]

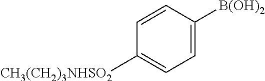

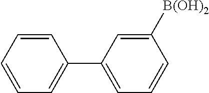 [SMILES: OB(O)c1cccc(-c2ccccc2)c1]

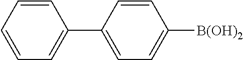 [SMILES: OB(O)c1ccc(-c2ccccc2)cc1]

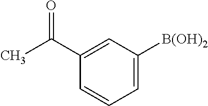

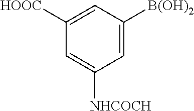

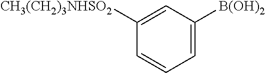

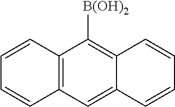 [SMILES: OB(O)c1c2ccccc2cc2ccccc12]

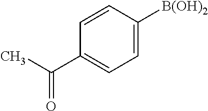

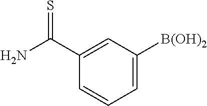 [SMILES: NC(=S)c1cccc(B(O)O)c1]

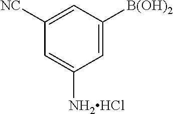

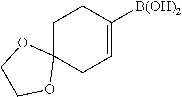 [SMILES: OB(O)C1=CCC2(CC1)OCCO2]

The present methods, compounds, or compositions may be full agonist, partial agonist, antagonist, inverse agonist, or differential agonists (i.e. have differential modes of binding) at one or more receptors described herein (e.g. one or more serotonin receptors). The methods can provide for differential yet selective activation at one or more receptors described herein (e.g. one or more serotonin receptors). The present methods, compounds, or compositions do not necessitate providing full agonists at one or more serotonin receptors described herein. The dual partial agonist can selectively bind to serotonin 5-HT₇ receptor and/or serotonin HT_{1A} receptor with a binding affinity (K) of less than about 100 nM, or less than about 50 nM, or less than about 25 nM, or less than about 20 nM, or less than about 10 nM, or less than about 5 nM, or less than about 2 nM, or less than about 1 nM. The dual partial agonist can provide selective binding to one or more of the serotonin 5-HT₇ and 5-HT_{1A} receptors, for instance providing at least about 10-fold, or at least about 20-fold, or at least about 30-fold, or at least about 40-fold, or at least about 50-fold, or at least about 75-fold, or at least about 100-fold higher affinity than one or more of the serotonin 5-HT_{2A} and 5-HT_{2C} receptors. For some dual partial agonists, the dual partial agonist does not bind one or more of the histamine H1 receptor, dopamine D2, and adrenergic α_{1A} and α_{1B} receptors at levels less than about 100 nM, or less than about 50 nM, or less than about 10 nM, or less than about 5 nM, or less than about 1 nM.

As used herein, the term “heteroalkyl” refers to an alkyl group connected to a molecule though a carbon atom of the chain, wherein one or more other (non-binding) carbon atoms may be substituted by a non-carbon and non-hydrogen atom, for example O, P, B, N, S, or Si. As used herein, the term “halo” refers to F, Cl, Br, or I.

### Examples

### Example 1. Syntheses

All commercially available regents and solvents were purchased and used without purification, unless otherwise specified. Flash column chromatography was performed with the use of Agela Technologies 230-400 mesh silica gel. Analytical thin-layer chromatography (TLC) was carried out on Agela Technologies silica gel 60 F254 plates. Final compounds were used as free base or their corresponding HCl salts, as noted below, depending on ease of manipulation of the free base. All spectra were recorded by a Varian 500 MHz or 400 MHz NMR in CDCl₃ or CD₃OD as noted and are expressed as chemical shift (δ) values in parts per million (ppm). Coupling constants (J) are presented in Hertz. Abbreviations used in the reporting of NMR spectra include, s=singlet, bs=broad singlet, d=doublet, t=triplet, q=quartet, quin=quintet, dd=doublet of doublets, qd=quartet of doublets, dt=doublet of triplets, tt=triplet of triplets, ddd=doublet of doublets of doublets, ddt=doublet of doublet of triplets, dtt=doublet of triplet of triplets, m=multiplet). High resolution mass spectrometry (HRMS) was performed with Waters Q-TOF Ultima ESI instrument using time of flight (TOF-MS) and electron spray ionization (ESI) or using a Waters 70-VSE instrument using time of flight (TOF-MS) and electron ionization (El). In some examples, both chiral synthesis and achiral synthesis, followed by chiral prep. chromatography, was utilized. HPLC separation of (+) and (−) enantiomers was determined by UV Trace 220/254 nm on a ShimadzuTM instrument equipped with a semi-preparative (s-prep)-RegisCelITM (5 μm, 25 cm×10 mm i.d.) chiral (polysaccharide-based) column. Optical rotation was determined using a JACSO (P-2000) polarimeter. Purity of targeted compounds was >95% (determined by HPLC) unless otherwise noted.

### Compound (2S)-5-PT: N,N-dimethyl-5-phenyl-1,2,3,4-tetrahydronaphthalen-2-amine HCl

Obtained from Scheme 2 (0.5 mmol) as a clear oil, yield; 60% (0.075 g), purification 95:5 (DCM:MeOH). The oil was converted to the corresponding HCl salt. ¹H NMR (500 MHz; CDCl₃): 8 12.64 (s, 1H), 7.41 (t, J=7.3 Hz, 2H), 7.35 (t, J=7.3 Hz, 1H), 7.23 (t, J=8.3 Hz, 3H), 7.15 (d, J=7.5 Hz, 1H), 7.12 (d, J=7.4 Hz, 1H), 3.54 (s, 1H), 3.39 (dd, J=15.4, 3.9 Hz, 1H), 3.20 (dd, J=14.8, 11.8 Hz, 1H), 2.85-2.79 (m, 8H), 2.40 (d, J=10.9 Hz, 1H), 1.81 (qd, J=11.4, 6.6 Hz, 1H). 13C NMR (400 MHz; CDCl₃): 8 142.2, 141.0, 132.34, 132.28, 129.1, 128.72, 128.63, 128.4, 127.4, 126.7, 62.3, 39.8, 39.5, 30.1, 27.3, 24.3 HRMS: Calc'd 5 C18H21N for [M+H]+: 252.1752. Found: 252.1756. HPLC (s-prep): Solvent System: hexanes/EtOH (91:1) 0.1% DEA (modifier) 0.1% TFA (modifier); flow rate=3.0 mL/min; t1=16.5, t2=18.1 (+) HCl: [a]D²⁵+36.6 for (2R), (c 0.2, MeOH), (−) HCl: [a]D²⁵-36.94 for (2S), (c 0.3, MeOH).

### Compound (2S)-5-FPT: (2S)-5-(2-fluorophenyl)-N,N-dimethyl-1,2,3,4-tetrahydronaphthalen-2-amine HCl

The product after step ‘c’ in Scheme 2 (0.13 g, 0.4 mmol) was dissolved in 3 mL di-methoxyethane and 0.3 mL water. The solution was degassed with N₂ and 0.112 g (0.8 mmol) K₂CO₃ was added followed by 0.062 g (0.44 mmol) of 2′-F-boronic acid and 0.071 g (0.06 mmol) Pd[PPh₃]₄. The reaction stirred for 4 hours at reflux and was quenched with water and extracted with ethyl acetate (3×20 mL) and organic fractions were dried over sodium sulfate. Purification was done by flash chromatography 95:5 (DCM:MeOH) to yield 0.03 g (23%) as a yellow oil. The oil was converted to the corresponding HCl salt. TLC eluents used (9:1 Dichloromethane: MeOH). ¹H NMR (500 MHz; CDCl₃): 8 11.82 (s, 1H), 7.37 (td, J=9.3, 4.3 Hz, 1H), 7.27-7.24 (m, 1H), 7.20 (t, J=7.0 Hz, 3H), 7.13 (t, J=8.0 Hz, 2H), 3.56-3.53 (m, 1H), 3.38-3.35 (m, 1H), 3.19 (t, J=12.7 Hz, 1H), 2.86-2.80 (m, 7H), 2.70-2.67 (m, 1H), 2.38 (d, J=10.2 Hz, 1H), 1.85-1.83 (m, 1H). 13C NMR (400 MHz, CD₃OD): 8 133.52, 131.33, 129.64, 129.55, 129.21, 128.53, 126.26, 126.25, 124.36, 124.30, 115.47, 62.54, 39.24, 29.85, 26.45, 23.98. Calc'd C18H20FN for [M+H]+: 270.1658. Found: 270.1656. (2S)-5-FPT HCl: [a]D²⁵-41.2 (c 0.7, DCM).

### Compound (2S)-5-CPT: (2S)-5-(2-chlorophenyl)-N,N-dimethyl-1,2,3,4-tetrahydronaphthalen-2-amine HCl

The product after step ‘c’ in Scheme 2 (0.065 g, 0.2 mmol) was dissolved in 1 mL dioxane. The solution was degassed with N₂ and 0.068 g (0.3 mmol) K₃PO₄ was added followed by 0.038 g (0.24 mmol) of 2′-Cl-boronic acid, 0.026 g KBr (0.22 mmol) and 0.023 g (0.02 mmol) Pd[PPh₃]₄. The reaction stirred for 6 hours at reflux and was quenched with water and extracted with ethyl acetate (3×20 mL) and organic fractions were dried over sodium sulfate. Purification was done by flash chromatography 95:5 (DCM:MeOH) to yield 0.04 g (61%) as a yellow oil. The oil was converted to the corresponding HCl salt. TLC eluents used (9:1 Dichloromethane: MeOH). ¹H NMR (500 MHz; CDCl₃): 8 12.49 (s, 1H), 7.45 (m, 1H), 7.32 (dd, J=8.3, 4.4 Hz, 2H), 7.23 (t, J=7.0 Hz, 1H), 7.19-7.14 (m, 2H), 7.03 (dd, J=6.7, 4.4 Hz, 1H), 3.53-3.49 (m, 1H), 3.39 (s, 1H), 3.22 (t, J=12.4 Hz, 1H), 2.86 (s, 6H), 2.73-2.52 (m, 2H), 2.40-2.38 (m, 1H), 1.87 (t, J=5.4 Hz, 1H). 13C NMR (400 MHz, CDCl₃): δ 139.71, 139.43, 139.40, 139.16, 133.42, 132.93, 132.79, 132.72, 132.19, 131.95, 131.01, 130.65, 129.57, 129.53, 129.46, 129.28, 129.20, 129.04, 128.26, 127.97, 126.97, 126.91, 126.44, 62.33, 62.06, 40.03, 39.72, 30.17, 29.97, 26.74, 26.01, 23.94, 23.84. Calc'd C18H20CIN for [M+H]+: 286.1363. Found: 286.1368. (2S)-5-CPT HCl: [a]D25-45.5 (c 0.5, MeOH).

### Compound (2S)-FPyT: 1-(5-(2-fluorophenyl)-1,2,3,4-tetrahydronaphthalen-2-yl)-pyrrolidine

Obtained from Scheme 1 (0.62 mmol) as a red oil, yield: 69% (0.11 g), purification 9:1 (DCM:MeOH); ¹H NMR (500 MHz; CDCl₃): 8 7.20-7.15 (m, 2H), 7.10 (dd, J=6.6, 2.1 Hz, 1H), 6.72 (t, J=2.2 Hz, 1H), 6.23 (dd, J=3.4, 2.8 Hz, 1H), 6.07 (dd, J=3.5, 1.8 Hz, 1H), 3.41 (s, 3H), 3.07-3.02 (m, 1H), 2.85 (dd, J=15.9, 10.7 Hz, 1H), 2.73-2.67 (m, 1H), 2.64 (tdd, J=10.8, 5.1, 3.1 Hz, 1H), 2.56 (ddd, J=22.0, 10.7, 4.9 Hz, 1H), 2.39 (s, 6H), 2.10-2.05 (m, 1H), 1.55 (qd, J=11.9, 5.2 Hz, 1H). 13C NMR (400 MHz, CDCl₃): 8136.95, 136.42, 132.96, 132.75, 129.47, 128.86, 125.36, 121.63, 108.41, 107.27, 60.66, 41.88, 34.20, 32.73, 27.71, 26.25. Calc'd C17H22N₂ for [M+H]+: 255.1861. Found: 255.1853. HPLC (s-prep): Solvent System: hexanes/EtOH (85:15) 0.1% DEA (modifier) 0.1% TFA (modifier); flow rate=2.0 mL/min; t1 =19.1, t2=22.5. (+)-5-PyT: [a]D²⁵+10.5 (c 0.3, MeOH), (—)-5-PyT: [a]D25 −14.3 (c 0.3, MeOH). 2S stereochemistry was assigned based on elution time of other known S-compounds from the HPLC chiral column.

### Compound (2S)-5-Na T: (2S)—N,N-dimethyl-5,6,7,8-tetrahydro-[1,1′-binaphthalen]-6-amine

The product after step ‘c’ in Scheme 2 (0.060 g, 0.19 mmol) was dissolved in 1.5 mL di-methoxyethane and 0.15 mL water. The solution was degassed with N₂ and 0.101 g (0.73 mmol) K₂CO₃ was added followed by 0.126 g (0.73 mmol) of 1-naphthyl-boronic acid and 0.033 g (0.03 mmol) Pd [PPh₃]₄. The reaction stirred for 4 hours at reflux and was quenched with water and extracted with ethyl acetate (3×20 mL) and organic fractions were dried over sodium sulfate. Purification was done by flash chromatography 95:5 (DCM:MeOH) to yield 0.03 g (53%) as a yellow oil. TLC eluents used (9:1 Dichloromethane: MeOH). ¹H NMR 1H NMR (400 MHz; CDCl₃): 8 7.80 (d, J=8.2 Hz, 1H), 7.76 (d, J=8.2 Hz, 1H), 7.44-7.35 (m, 2H), 7.30-7.27 (m, 1H), 7.22 (dd, J=13.8, 7.0 Hz, 1H), 7.17-7.11 (m, 2H), 7.00 (t, J=8.8 Hz, 1H), 3.07-2.99 (m, 1H), 2.83 (dt, J=16.9, 9.0 Hz, 1H), 2.67-2.58 (m, 1H), 2.39 (td, J=11.4, 4.2 Hz, 1H), 2.31 (d, J=3.8 Hz, 7H), 2.25-2.19 (m, 1H), 1.90-1.82 (m, 1H), 1.46-1.35 (m, 1H). 13C NMR (400 MHz, CDCl₃): δ 140.33, 139.99, 138.77, 138.36, 133.59, 133.43, 133.25, 133.20, 132.07, 131.82, 131.76, 131.64, 129.16, 128.92, 128.89, 128.40, 128.30, 127.83, 127.78, 126.78, 126.54, 126.41, 126.35, 126.29, 126.16, 125.92, 125.89, 125.67, 125.44, 125.34, 62.54, 62.46, 46.28, 30.47, 30.36, 26.81, 26.34, 24.07, 23.70. Calc'd C22H23N for [M+H]⁺: 302.1909. Found: 302.1909. (2S)-5-NaT: [a]D²⁵-59.5 (c 0.5, DCM).

### Compound (2S)-5-PPT: (2S)-5-phenyl-N,N-dipropyl-1,2,3,4-tetrahydronaphthalen-2-amine HCl

In Scheme 2 were implemented propionaldehyde, montmolillonite K 10, NaBH3CN, MeOH, rt, 20 h, (c)N-(2-pyridyl)-bis-(trifluoromethanesulfonimide), DIPEA, CH2C12, 0 C to It, 20 h; product at (0.13 mmol) to yield 0.026 g (67%) as a clear oil, purification 98:2 (DCM:MeOH). The oil was converted to the corresponding HCl salt. ^{1H} NMR (500 MHz; CDCl₃): 8 12.02 (s, 1H), 7.41 (t, J=7.3 Hz, 2H), 7.35 (t, J=7.1 Hz, 1H), 7.23 (dd, J=12.5, 7.6 Hz, 3H), 7.15 (d, J=7.5 Hz, 1H), 7.12 (d, J=7.4 Hz, 1H), 3.68 (s, 1H), 3.39-3.36 (m, 1H), 3.28 (t, J=13.0 Hz, 1H), 3.09 (dd, J=15.9, 13.0 Hz, 2H), 2.98 (t, J=5.4 Hz, 2H), 2.80 (d, J=5.1 Hz, 2H), 2.45 (d, J=10.9 Hz, 1H), 2.05 (s, 2H), 1.95 (d, J=4.5 Hz, 2H), 1.86 (dd, J=19.3, 10.7 Hz, 1H), 1.02 (q, J=5.3 Hz, 6H). 13C NMR (400 MHz, CDCl₃): δ 142.19, 141.06, 132.59, 132.53, 129.13, 128.70, 128.58, 128.41, 127.37, 126.62, 59.69, 52.45, 52.09, 30.29, 27.61, 24.53, 18.16, 11.63. Calc'd C22H29N for [M+H]⁺: 308.2378. Found: 308.2376. (2S)-5-PPT HCl: [a]D25 −33.14 (c 0.5, MeOH).

### Compound (2S)-5-CPPT: (2S)-5-(2′-chlorophenyl)-N,N-dipropyl-1,2,3,4-tetrahydronaphtha-len-2-amine

In Scheme 2 were implemented propionaldehyde, montmolillonite K 10, NaBH3CN, and MeOH, rt, 20 hours. Obtained from (2S)-precursor (0.17 mmol) to yield 0.01 g (21%) as a light yellow oil, purification 97:3 (DCM:MeOH). ¹H NMR (500 MHz; CDCl₃): 8 7.46 (m, 1H), 7.31 (m, 2H), 7.22 (m, 1H), 7.18 (m, 2H), 7.02 (t, J=5.6 Hz, 1H), 3.59-3.44 (m, 1H), 3.29-3.15 (m, 2H), 3.00-2.82 (m, 4H), 2.68-2.50 (m, 2H), 2.34-2.23 (m, 1H), 1.93-1.77 (m, 5H), 0.98 (d, J=5.4 Hz, 6H). The oil was converted to the corresponding HCl salt. 13C NMR (400 MHz, 0D013): 8 139.72, 139.41, 139.34, 139.17, 133.44, 133.03, 132.86, 132.78, 132.45, 132.19, 130.99, 130.61, 129.51, 129.39, 129.14, 128.94, 128.17, 127.88, 126.86, 126.82, 126.31, 59.52, 59.36, 52.57, 52.30, 52.27, 52.15, 51.95, 30.43, 30.02, 27.01, 26.23, 24.09, 18.26, 17.81, 11.53. Calc'd C22H28CIN for [M+H]⁺: 342.1989. Found: 342.1979. (2S)-5-CPPT HCl: [a]D25 −35.18 (c 0.25, MeOH).

### Compound (R/S)-5-FPyT

In Scheme 3 below, reagents and conditions were: (a) Dipropylamine HCl, NaBH3CN, MeOH/THF, 55 C, 16 h: (b) Pyrrolidine, NaBH3CN, MeOH/THF. 55 C, 16 h. The tetralone intermediate 7b underwent reductive amination with dipropylamine hydrochloride or pyrrolidine to give the racemic products. Racemate 17 was successfully resolved by chiral-HPLC while attempts to resolve 18 were unsuccessful.

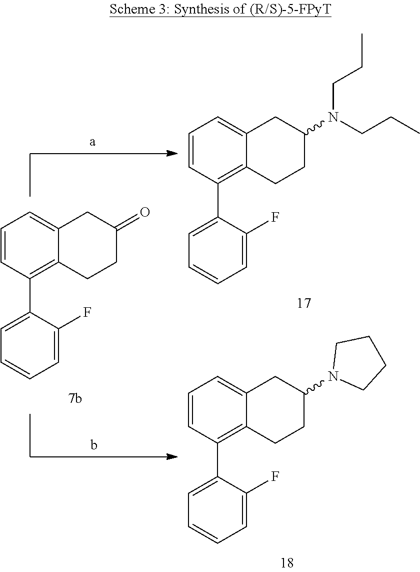 [SMILES: O=C1CCc2c(cccc2-c2ccccc2F)C1 ; Fc1ccccc1-c1cccc2c1CCC(N1CCCC1)C2 ; CCCN(CCC)C1CCc2c(cccc2-c2ccccc2F)C1]

### Example 2. Computational Chemistry and Molecular Modeling

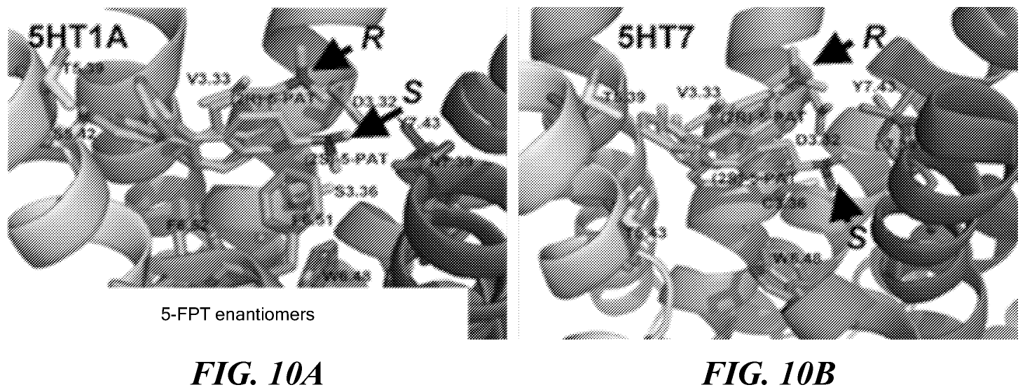

Molecular models of the human 5HT1A and 5HT7 GCPRs were built by homology to the recent 5HT₁B crystal structure (pdb code: 4IAQ). Molecular docking and molecular dynamics (MD) simulations on selected 5-PAT analogs was done for which experimentally-determined affinity and functional data is known (Table 1, FIGS. 2-3) to help understand molecular determinants for binding and function at 5HT1Ain comparison to 5HT7 receptors. Molecular dynamics simulations and docking experiments conducted with (2R)- and (2S)-5-FPT indicated a low energy pose of (2S)-5-FPT (green) in complex with the 5HT1A receptor in FIG. 10A such that the 2-dimethylamine moiety likely forms an ionic bond with D3.32 (2.86 Å) and hydrophobic interactions with Y7.43 (3.85 Å), F6.51 (3.81 Å), W6.48 (3.86 Å), C3.36 (3.55 Å) to result in high-affinity (Kᵢ=4 nM, Table 1) binding. In contrast, the preferred conformation of (2R)-5-FPT (light blue) docks such that it forms an ionic bond with D3.32 (2.74 Å) but does not form the hydrophobic interactions, likely, explaining its nil affinity (Kᵢ=1,000 nM). MD simulations followed by molecular mechanics-generalized Born surface area (MMGBSA) binding free energy calculations suggested the (2S)-5-FPT has low overall binding free energy (−40.66 kcal/mol) at 5HT1A compared with (2R)-5-FPT (−37.26 kcal/mol), which is consistent with experimentally-determined affinity values. Fiα. 10B shows that (2S)-5-FPT (green) binds differently at 5HT7 compared with (2R)-5-FPT, i.e., the 2-dimethylamine amine moiety binds deeper into the binding pocket, forms an ionic bond with D3.32 (2.87 Å) and hydrophobic interactions with Y7.43 (3.77 Å), L7.39 (3.63 Å) and C3.36 (3.89 Å); the 5-phenyl and 2-aminotetralin groups form hydrophobic interactions with F6.51 (3.69 Å), V3.33 (3.64 Å), F6.52 (3.48 Å), S5.42 (3.34 Å), A5.46 (3.60 Å), Y5.38 (3.76 Å), 15.32 (3.59 Å), G7.42 (3.33 Å) to result in higher affinity (K=5 nM, Table 1). In contrast, the (2R)-5-FPT (light blue) forms an ionic bond with D3.32 (2.76 Å) and hydrophobic interactions with only L7.39 (3.54 Å), F6.52 (3.60 Å), T5.43 (3.58 Å), L5.31 (3.54 Å), C5.30 (3.88 Å), V3.33 (3.47 Å), likely, explaining its lower affinity (Kᵢ=750 nM). The calculated MMGBSA binding free energies are −40.67kca1/mol and −37.16 kcal/mol for (2S)-5-FPT and (2R)-5-FPT, respectively, consistent with results.

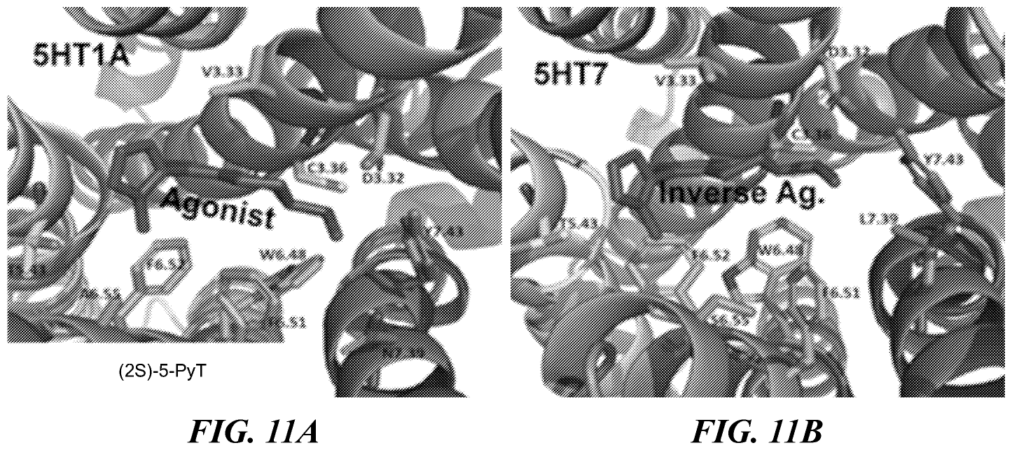

FIG. 11A shows the dock of (2S)-5-PyT at the 5HT1A receptor, where it is an agonist, and, FIG. 11B shows (2S)-5-PyT docked at 5HT7 where it is an inverse agonist. The 2-dimethylamine moiety of ligand forms an ionic bond with D3.32, and, forms numerous (12) hydrophobic interactions with 5HT1A residues, i.e., Y7.43, S5.33, Y5.38, F6.52, C3.36, W6.48, F6.51, V3.33, T5.39, A6.55, A5.46, S5.42. In contrast, at 5HT₇, (2S)-5-PyT does not interact with W6.48 and F6.51 (highly-conserved among aminergic GPCRs and impact intracellular loop 3 interaction with G-protein) and forms different and fewer (8) overall hydrophobic interactions, i.e., Y7.43, S5.33, Y5.38, F6.52, C3.36, L7.39, 15.32, T5.43. Importantly, the hydrophobic interaction (3.3 Å) between the pyrrole methyl group of (2S)-5-PyT and A6.55 in 5HT1A(FIG. 3) is absent at 5HT7 where there is serine in the position (S6.55, FIG. 4). This difference likely plays a role in determining agonist (5HT1 Å) vs. inverse agonist (5HT₇) activity of the ligand.

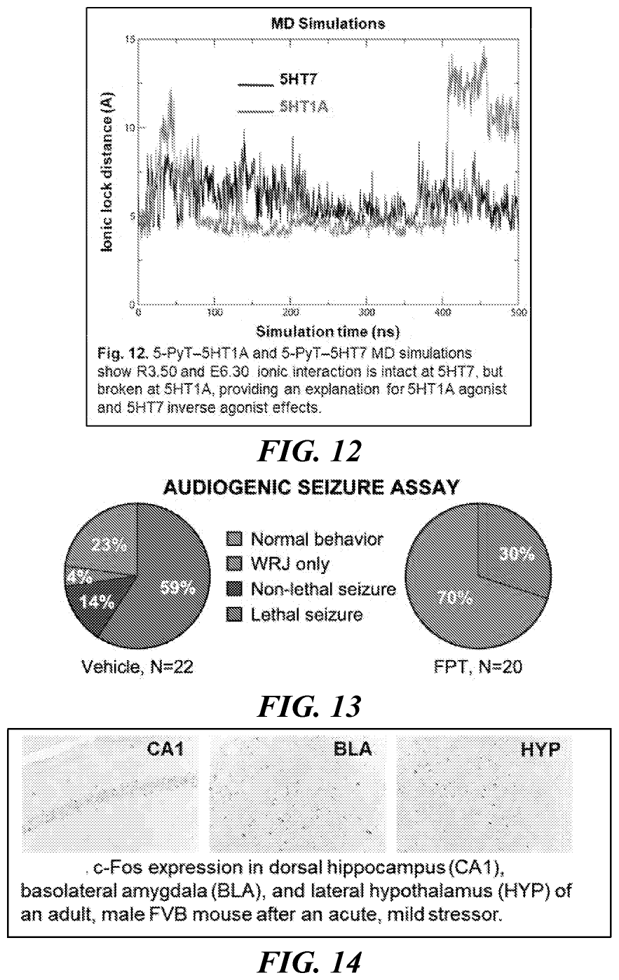

Additional MD simulations (run-time 500 ns) of (2S)-5-PyT interacting with the 5HT1A or 5HT7 receptor were performed to model the ionic lock distance between R^{3.50} and E6.30—a key interaction that restrains aminergic GPCRs in an inactive conformation). FIG. 12, shows that for 5HT1A, during the time 400-500 ns, the ionic lock is not apparent, as the distance between R3.50 and E6.30 increased to >10 Å, i.e., (2S)-5-PyT stabilized an active conformation of 5HT1A. In contrast, during the 500 ns MD run for 5HT₇, the distance between R3.50 and E6.30 remained ˜5 Å, suggesting ionic interaction, i.e., (2S)-5-PyT stabilized an inactive conformation of 5HT7. This result gives confidence that MD simulations will help lead to understanding of molecular determinants for 5-PAT interactions with 5HT₁ and 5HT7 that lead to agonist vs inverse agonist functional activity.

Exploiting 5-PAT-Binding Pocket Differential Interactions at 5HT1Avs. 5HT7: Although there is close TMD homology between 5HT7 and 5HT1A, there are important differences in TMDs 6 and 7, e.g., positions 6.55 and 7.39. Position 6.55 is alanine (A) in 5HT1A and serine (S) in 5HT7. Meanwhile, position 7.39 is asparagine (N) in 5HT1A and leucine (L) in 5HT7. Preliminary docking results above show that (2S)-5-FPT and (2S)-5-PyT dock close to TMDs 6 and 7, indicating, modification to the 5-PAT scaffold, particularly at the C(5)-position, likely will impact affinity selectively between 5HT7 and 5HT1A, as well as, agonist vs. inverse agonist functional activity. Current modeling results can be validated, iteratively, using point-mutated receptors, e.g., A6.55S 5HT1A, S6.55A 5HT7, N7.39L 5HT1A, L7.39N 5HT7. Analogs for synthesis shown below include substitutions at the C(5)-position, hypothesized to differentially interact with 5HT7 and 5HT1A residues in TMDs 6 and 7. The ligand design process is an iterative modus operandi involving molecular docking studies, medicinal chemical synthesis, pharmacological assessment, including using point-mutated receptors to validate proposed ligand-receptor interactions governing receptor affinity, selectivity and function.

### Example 3: (2S)—S-FPT Affinity at 5-HT₇ and 5-HT_{1A} and Partial Agonism

HEK293 cells stably expressing human 5-HT₇ receptors were generated to assess 5-HT₇ pharmacology of 5-FPT enantiomers. Receptor binding site density in the clone with the highest specific binding (“CHTR7beta”) was assessed with [³H]5-CT saturation binding, which revealed a mean (SEM) receptor binding site density, B_{MAX}, of 7.7 (0.4) pmol/mg protein. At such a high 5-HT₇ receptor density, a 5-HT₇ partial agonist is not expected to appear as a full agonist, because receptor reserve is not an issue. In addition, 5-FPT pharmacology was evaluated in HEK293 cells transiently over-expressing relevant human 5-HT receptors (5-HT_{1A, 2A, 2B, 2C}), and potential ‘off-targets’, including the dopamine D2, adrenergic α_{1A, 1B,} and histamine H₁ receptors. Notably, D2 can display high affinity for the 2-aminotetralin scaffold, depending on substitution pattern and stereochemistry, and α₁ and H₁ receptors are common off-targets of antipsychotics used to treat irritability in ASD. Studies also were conducted using HEK293 cells transiently expressing the mouse 5-HT_{2A} and 5-HT_{2C} receptors, given their relevance to the translational studies.

As shown in Table 1, 5-FPT demonstrated high, stereoselective affinity at 5-HT₇ receptors, and the (S)-enantiomer (kᵢ=4±0.1) was about 80-times more potent than the (R)-enantiomer (Kᵢ=˜460 nM). (2S)—S-FPT behaved as a 5-HT₇ partial agonist regarding Gs-cAMP signaling (EC50=34 nM, E_{MAX} vs AS-19=33%) and the (R)-enantiomer also demonstrated partial agonism (EC50=378 nM, E_{MAX} vs AS-19=29%) with similar kinetics, albeit, with about 11-times less potency than the (S)-isomer (FIGS. 2-3).

### Example 4: (2S)—S-FPT Attenuates Motor Stereotypy without Affecting Locomotion

(2S)—S-FPT was tested in three, heterogeneous models of stereotypy, each with different scales of validity: 1) idiopathic stereotypic jumping in C58/J mice (FIG. 4); 2) (±)-2,5-dimethoxy-4-iodoamphetamine (DOI)-elicited stereotypic head-twitching (FIG. 5); 3) and (5R,10S)-(+)-5-methyl-10,11-dihydro-5H-dibenzo[a,d]cyclohepten-5,10-imine (MK-801) elicited stereotypic rotations in C57BI/6J mice (FIG. 6). (2S)-5-FPT was also tested for efficacy to attenuate d-amphetamine (AMP)-elicited hyperlocomotion in C57BI/6J mice to assess potential pharmacological utility for psychostimulant abuse. 058/J mice naturally develop repetitive, stereotypic jumping when housed under standard laboratory conditions, and this behavior has been used as a model of stereotypy responsive to drug treatment. As shown in FIG. 4, (2S)-5-FPT potently eliminated stereotypic jumping in 058/J mice in a dose-dependent manner, without altering locomotor behavior (see FIG. 8). (2S)-5-FPT showed greater efficacy in this model than the recently reported mGluR5 negative allosteric modulator, GRN-529, which was under development to treat ASD.

For measurement of marble burying (FIG. 7), FVB mice were placed individually, for 10 min, in clean boxes (6 in. ×10 in. ×5 in.) containing 18 clear marbles atop fresh home-bedding. Marbles buried (i.e., marbles 50% covered with bedding) were calculated after the test session.

Regarding glutamate neurotransmission in stereotypy, the NMDA receptor antagonist, MK-801, characteristically elicits stereotypic rotations that appear to mimic monogenetic stereotypy observed in Fmr1 KO mice. Furthermore, mutations and autoantibodies of the NMDA glutamate receptor that decrease its function are causally linked to ASD, intellectual disabilities, and psychiatric symptoms in humans. As shown in FIG. 6, (2S)-5-FPT (5.6 mg/kg) significantly reduced stereotypical rotations in C57BI/6J mice treated with MK-801. Note, neither (2S)-5-FPT nor AMP caused stereotypic rotational behavior (FIG. 6). Additionally, (2S)-5-FPT (5.6 mg/kg) significantly decreased hyperlocomotion caused by MK-801, but did not reduce hyperlocomotion caused by AMP. Importantly, on its own, (2S)-5-FPT also did not alter locomotion in C57BI/6J mice.

The DOI-elicited HTR is a behavioral model of cortical 5-HT_{2A} activation, and also has face validity for stereotyped tics. The 5-HT_{2A} receptor is a predominant 5-HT receptor in the cortex, and serves important excitation modulation functions on glutamate pyramidal and GABA neurons. 5-HT_{2A} receptor function in cortical neurons is altered in Fmr1 KO mice, and 5-HT_{2A} function also is disrupted in persons with ASD and Tourette syndrome. Moreover, 5-HT_{2A} antagonists such as ketanserin (tritiated version used here as the 5-HT_{2A} radiolabel) treat tics in Tourette syndrome, and when infused in subthalamic nuclei, reduce stereotypies in rats, supporting the DOI-elicited HTR as a model of stereotypy and/or tics. Relevant, too, the 5-HT_{1A} receptor partial agonist buspirone, in clinical trials to treat children with ASD, has clinically, germane affinity (Kᵢ˜140 nM) at 5-HT_{2A} receptors. As shown in FIG. 6, (2S)-5-FPT dose-dependently attenuated the DOI-elicited HTR, and significant attenuating effects were observed with each dose. Notably, DOI has weak affinity at both 5-HT_{1A} and 5-HT₇ receptors (Kᵢ>1 μM, unreported observations), and (2S)-5-FPT has weak activity at 5-HT_{2A} receptors, which mediate the DOI-elicited HTR in C57BI/6J mice, suggesting the effect of (2S)-5-FPT was not due to competition with DOI for receptor sites, but was indirectly modulating DOI-elicited 5-HT_{2A} receptor activity to impact behavior. Importantly, although (2S)-5-FPT showed weak partial agonist activity at HEK cells over-expressing human 5-HT_{2A} receptors, it did not elicit an HTR on its own (Table 3). To further support the assertion that (2S)-5-FPT reduced the DOI HTR via receptor mechanisms other than 5-HT_{2A}, tests of (2R)-5-FPT, AS-19, (+)-DPAT, and (—)-DPAT in this assay were also conducted. The (2R)-5-FPT enantiomer that has the same physicochemical properties as (2S)-5-FPT, but substantially higher affinity at human and mouse 5-HT_{2A} receptors, with neutral antagonist function, was substantially less efficacious than (2S)-5-FPT at the 5.6 mg/kg dose to reduce the HTR. Furthermore, AS-19 and both enantiomers of DPAT, all of which have weak affinity at 5-HT_{2A}, 5-HT_{2B}, and 5-HT_{2C} receptors, but, clinically relevant affinity at 5-HT₇ and 5-HT_{1A} receptors, suppressed the DOI HTR. Notably, (+)-DPAT, a 5-HT_{1A} full agonist caused severe hypolocomotion and obvious serotonin syndrome in this assay, whereas neither (—)-DPAT, a 5-HT_{1A} partial agonist, nor AS-19 affected locomotor behavior or caused obvious serotonin syndrome (data not shown). Overall, the results support previous assertions that DPAT, via 5-HT_{1A} activation, suppresses the DOI HTR, and also suggest that 5-HT₇ receptor activation may additionally contribute to the effect, which may translate as suppression of stereotyped behaviors. Furthermore, relative to full agonists, 5-HT_{1A} partial agonists appear to translate with fewer untoward effects, such as behaviors associated with serotonin syndrome.

### Example 5: (2S)-5-FPT Increases Social Interactions and does not Cause Symptoms of Serotonin Syndrome

As shown in FIG. 9, (2S)-5-FPT (5.6 mg/kg) significantly increased the number of initiated social interactions in C57BI/6J mice, while also decreasing grooming. Furthermore, as shown in Table 2, (2S)-5-FPT, at the highest behaviorally-effective dose tested (5.6 mg/kg), did not result in symptoms of serotonin syndrome, including flat body, forepaw treading, moon walking, piloerection, Straub tail, or tremor, but did significantly decrease rearing, suggestive of 5-HT_{1A} activation. After behavioral testing was complete, blind scorers categorized mice into two groups based on number of rears and initiated social interactions, and the two groups differentiated vehicle from (2S)-5-FPT-treated mice with 100% accuracy. In Table 2, shown are mean (SEM) number of session, from a total of six, one-minute observation session, in which mice displayed the behavior (score/6 possible), except for (n)=mean of the total number of responses across all six sessions.

### Example 6: (2S)-5-FPT is Orally Active And Readily Crosses the Blood-Brain Barrier

As shown in FIG. 5, (2S)-5-FPT significantly attenuated the DOI elicited head-twitching response (HTR) after administration. In addition, (2S)-5-FPT readily crosses the blood-brain barrier, as evidenced by detection of μg levels 30, 60, and 90 minutes after systemic administration (Table 4). Notably, levels of (2S)-5-FPT were substantially lower in plasma relative to brain tissue as soon as 30 minutes post-administration, indicating that (2S)-5-FPT is rapidly cleared in the periphery. Meanwhile, the attenuating effects of (2S)-5-FPT (5.6 mg/kg) on the DOI HTR remained significant for up to 2 hours post-administration; at 3 hours post-administration, (2S)-5-FPT did not block the DOI HTR.

Given the poor affinity of (2S)-5-FPT at D2 receptors, and the observations that (2S)-5-FPT (5.6 mg/kg) did not significantly decrease hyperlocomotion elicited by amphetamine, and without wishing to be bound by theory, (2S)-5-FPT may not be working directly through dopaminergic mechanisms. Because of (2S)-5-FPT's high affinity at 5-HT_{1A} and 5-HT₇ receptors, and because (2S)-5-FPT considerably affected behavior elicited by MK-801 and DOI, without wishing to be bound by theory, (2S)-5-FPT may work in vivo via 5-HT_{1A} and 5-HT₇ partial agonism mechanisms that regulates glutamatergic and/or 5-HT₂ receptor signaling. In Table 4 below, data are expressed as mean (SEM).

TABLE 4
Plasma and brain concentrations of (2S)-5-FPT
after 3.0 mg/kg subcutaneous administration.

Time after injection

| | 30 min | 60 min | 90 min |
|---|---|---|---|
| Plasma (μg/mL) | 0.114(0.03) | 0.118(0.01) | 0.070(0.01) |
| Brain (μg/g) | 1.78(0.24) | 2.16(0.17) | 1.46(0.09) |

### Example 7. (2S)-5-FPT Effects on FMr1 KO Mice

Fmr1 knockout mice are prone to audiogenic seizures that are elicited by continuous, high-decibel (e.g., 120 dB), high-frequency noise. Behaviors elicited by this auditory stimulus follow a sequential pattern that begins with a startle response soon after the noise onset, followed by freezing, then wild-running and jumping (WRJ) that can progress to tonic-clonic seizures and then to respiratory arrest. The entire sequence lasts for about 2 minutes. It was observed that juvenile (P23-P25) Fmr1 knockout mice were more susceptible to audiogenic seizures compared to adult (>P60) Fmr1 knockout mice (Fisher's exact test, P<0.0001). FIG. 13 (left) shows data from vehicle-treated, juvenile Fmr1 knockout mice (Jessica L. Armstrong, et al., 2020).

Mice treated with vehicle and mice treated with FPT were placed in adjacent, identical, clear polycarbonate boxes (18 in.×8 in.×8 in.), each covered with a plastic screen. After a 1 minute acclimation period, mice were exposed to 120 dB alarm (RadioShack Kit #49-1010, doorstop alarm) for 5 minutes, held by hand directly above the boxes. An audiogenic seizure was defined and categorized as a tonic-clonic seizure with the animal making a full recovery afterward or a tonic-clonic seizure progressing to respiratory arrest. WRJ, often observed before tonic-clonic seizures in mice, was also documented. In cases where mice did not exhibit any detectable alterations in behavioral activity, “normal behavior” was documented. Boxes were cleaned vigorously with running water and were towel dried after each test. FIG. 14 (right) shows data from (2S)-5-FPT treated, juvenile Fmr1 knockout mice.

A recent study had shown that absence of FMRP in the inferior colliculus is required for the audiogenic seizure phenotype in Fmr1 knockout mice. An examination was conducted of c-Fos expression in the inferior colliculus in juvenile Fmr1 knockout mice, pretreated with vehicle or FPT, that were exposed for 30 seconds to a 120 dB alarm. As shown in FIG. 15C, there was no effect of FPT on c-Fos expression in the inferior colliculus (P=0.63)

On the basis of the distribution of FMRP (Fragile X mental retardation protein) in the adult mouse brain and considering neural systems implicated in cognitive and neuropsychiatric symptoms (e.g., anxiety, sensory hypersensitivity, and social deficits) present in FXS and ASD, the effects of FPT were evaluated on c-Fos expression in the hippocampus (CA1, CA3, and dentate gyrus (DG)), the basolateral amygdala (ventral (BLAv), posterior (BLAp), and anterior (BLAa)), the somatosensory (SS) cortex, the hypothalamus (periventricular hypothalamic nucleus, intermediate (PVi), posterior hypothalamic nucleus (PH), and dorsomedial nucleus of the hypothalamus (DMH)), the paraventricular nucleus of the thalamus (PVT), and the ventral retrosplenial area (RSPv). Shown in FIG. 15A and FIG. 15B are results from analyses of c-Fos expression in CA3 and the BLAa, respectively. There were no main effects of the independent variables on c-Fos in CA3; however, there was a significant treatment effect in BLAa (F(1, 12)=14.22, P=0.0027). FPT increased c-Fos levels in the BLAa of Fmr1 knockout mice by 234% relative to vehicle, a statistically significant difference with a large effect (P=0.0046, d=2.18). In the BLAa of wild-type mice, FPT increased c-Fos expression by 157% relative to vehicle. The effect was large, but the difference was not statistically significant (P=0.0871, d=1.54). FPT did not significantly alter c-Fos expression in any other brain region examined; although, in every brain region assessed in Fmr1 knockout mice, the number of c-Fos positive cells was higher after FPT treatment.

1. A compound of Formula (I) 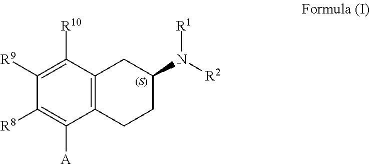
wherein A is selected from the group consisting of: 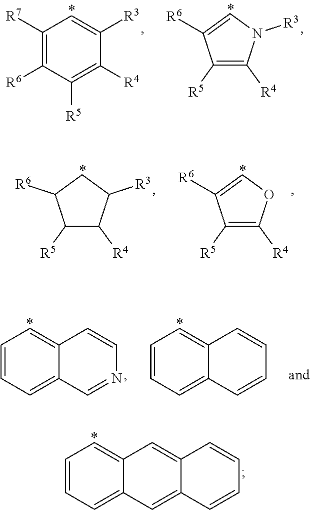
wherein A is attached to Formula (I) through carbon atom *;
wherein each of R¹ and R² is independently hydrogen or alkyl; and R¹ and R² may come together to form a substituted or unsubstituted alkyl ring, heterocyclic ring, or aromatic ring;
each of R³, R⁴, R⁵, R⁶, R⁷, R⁸, R⁹, and R¹⁹ is independently hydrogen, halo, hydroxy, acyl, acyloxy, alkyl, heteroalkyl, alkenyl, heteroalkenyl, alkynyl, heteroalkynyl, alkoxycarbonyl, carbonyl, cyano, sulfonamide, trifluoromethyl, trifluoromethoxy, nitro, amino, amido, and a cycloalkyl or cycloaryl, and wherein any two adjacent R groups may optionally come together to form a substituted or unsubstituted carbocyclic, aromatic, naphthalene, isoquinoline, or heterocyclic ring or ring system.

2. A compound selected from the group consisting of:

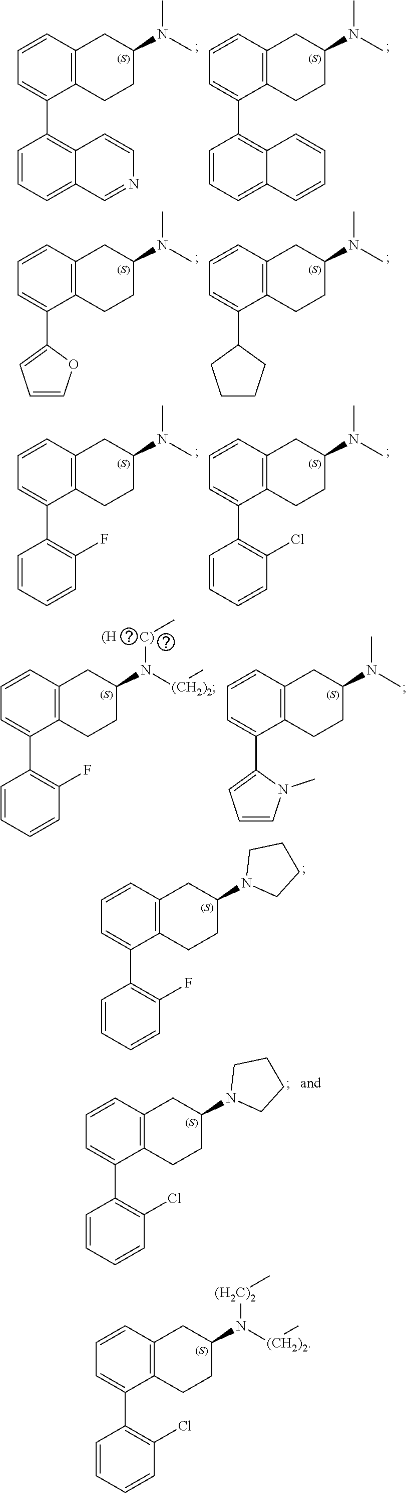

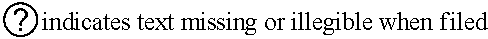

3. A pharmaceutical composition comprising a compound of claim 1 or claim 2, or a pharmaceutically acceptable salt, hydrate, or solvate thereof.

4. The pharmaceutical composition of claim 3, wherein the composition comprises at least about 70%, or at least about 80%, or at least about 85%, or at least about 90%, or at least about 95%, or at least about 97%, or at least about 99% of a single enantiomer of the compound.

5. The pharmaceutical composition of claim 3, wherein the compound binds to a serotonin 5-HT₇ receptor and/or a serotonin 5-HT_{1A} receptor with an affinity of less than about 100 nM, or less than about 50 nM, or less than about 25 nM, or less than about 20 nM, or less than about 10 nM, or less than about 5 nM, or less than about 2 nM, or less than about 1 nM.

6. The pharmaceutical composition of claim 3, wherein the compound is a dual partial agonist of the serotonin 5-HT₇ and serotonin 5-HT_{1A} receptors.

7. The pharmaceutical composition of claim 3, wherein the compound is an antagonist of a serotonin 5HT_{2B} receptor and/or a moderate affinity agonist of a serotonin 5HT_{2C} receptor.

8. The pharmaceutical composition of claim 3, wherein the compound binds to the serotonin 5-HT_{2B} receptor with a binding affinity of less than about 100 nM.

9. The pharmaceutical composition of claim 3, wherein the compound binds the serotonin 5-HT_{2A} receptor or the 5-HT_{2C} receptor with an affinity of greater than about 300 nM, or greater than about 400 nM, or greater than about 500 nM, or greater than about 750 nM, or greater than about 1 μM.

10. The pharmaceutical composition of claim 3, wherein the compound binds one or more of the serotonin 5-HT₇ and 5-HT_{1A} receptors with at least about 10-fold, or at least about 20-fold, or at least about 30-fold, or at least about 40-fold, or at least about 50-fold, or at least about 75-fold, or at least about 100-fold higher affinity than the affinity with which it binds to either the serotonin 5-HT_{2A} receptor or the serotonin 5-HT_{2C} receptor.

11. A method of treating or preventing a disease or disorder, comprising administering a therapeutically effective amount of an at least 70% pure (S)-enantiomer dual partial agonist at the serotonin 5-HT₇ and 5-HT_{1A} receptors, the dual partial agonist having the structure of Formula (I): 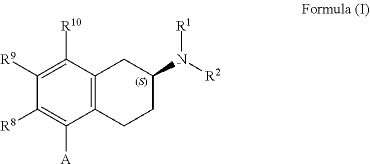
wherein A is selected from the group consisting of: 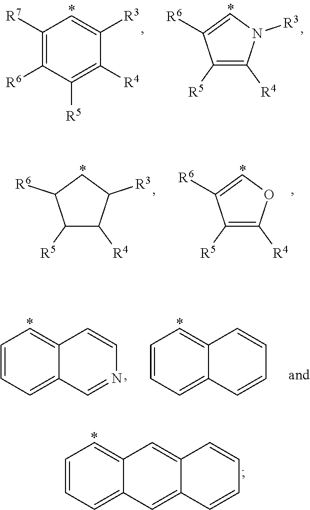
wherein A is attached to Formula (I) through carbon atom *;
wherein each of R¹ and R² is independently hydrogen or alkyl; and R¹ and R² may come together to form a substituted or unsubstituted alkyl ring, heterocyclic ring, or aromatic ring;
each of R³, R⁴, R⁵, R⁶, R⁷, R⁸, R⁹, and R¹⁹ is independently hydrogen, halo, hydroxy, acyl, acyloxy, alkyl, heteroalkyl, alkenyl, heteroalkenyl, alkynyl, heteroalkynyl, alkoxycarbonyl, carbonyl, cyano, sulfonamide, trifluoromethyl, trifluoromethoxy, nitro, amino, amido, and a cycloalkyl or cycloaryl, and wherein any two adjacent R groups may optionally come together to form a substituted or unsubstituted carbocyclic, aromatic, naphthalene, isoquinoline, or heterocyclic ring or ring system;
or a pharmaceutically acceptable salt, hydrate, or solvate thereof.

12. The method of claim 11, wherein the disease or disorder is selected from the group consisting of substance use disorder, opioid use disorder, addiction, anxiety, psychosis, depression, autism spectrum disorder, fragile X syndrome, neurological disorders, neuropsychiatric disorders, repetitive behaviors, movement disorders, compulsions, tics, pain disorders, vasospastic disorders, migraine headache, seizures, epilepsy, social anxiety, addiction withdrawal, drug withdrawal, drug abuse, alcoholism, eating disorders, general inflammation disorders, miosis, inflammatory bowel disease, ulcerative colitis, Crohn's disease, and gastrointestinal disorders.

13. The method of claim 11, wherein the disorder is substance use disorder caused by use of an opioid or THC.

14. The method of claim 11, wherein the dual partial agonist comprises at least about 70%, or at least about 80%, or at least about 85%, or at least about 90%, or at least about 95%, or at least about 97%, or at least about 99% of a single enantiomer of Formula (I).

15. The method of claim 11, wherein the dual partial agonist binds the serotonin 5-HT₇ and/or 5-HT_{1A} receptors with a binding affinity of less than about 100 nM, or less than about 50 nM, or less than about 25 nM, or less than about 20 nM, or less than about 10 nM, or less than about 5 nM, or less than about 2 nM, or less than about 1 nM.

16. The method of claim 11, wherein the dual partial agonist is an antagonist at the serotonin 5HT_{2B} receptor and/or a moderate affinity agonist at the serotonin 5HT₂C receptor.

17. The method of claim 11, wherein the dual partial agonist binds to the serotonin 5-HT₂B receptor with a binding affinity of less than about 100 nM.

18. The method of claim 17, wherein the dual partial agonist binds the serotonin 5-HT_{2A} and/or 5-HT_{2C} receptors with an affinity of greater than about 300 nM, or greater than about 400 nM, or greater than about 500 nM, or greater than about 750 nM, or greater than about 1 μM.

19. The method of claim 11, wherein the dual partial agonist binds one or more of the serotonin 5-HT₇ and 5-HT_{1A} receptors with at least about 10-fold, or at least about 20-fold, or at least about 30-fold, or at least about 40-fold, or at least about 50-fold, or at least about 75-fold, or at least about 100-fold higher affinity than the affinity with which it binds to either the serotonin 5-HT_{2A} receptor or the serotonin 5-HT_{2C} receptor.

20. The method of claim 11, wherein the dual partial agonist is a compound selected from the group consisting of: 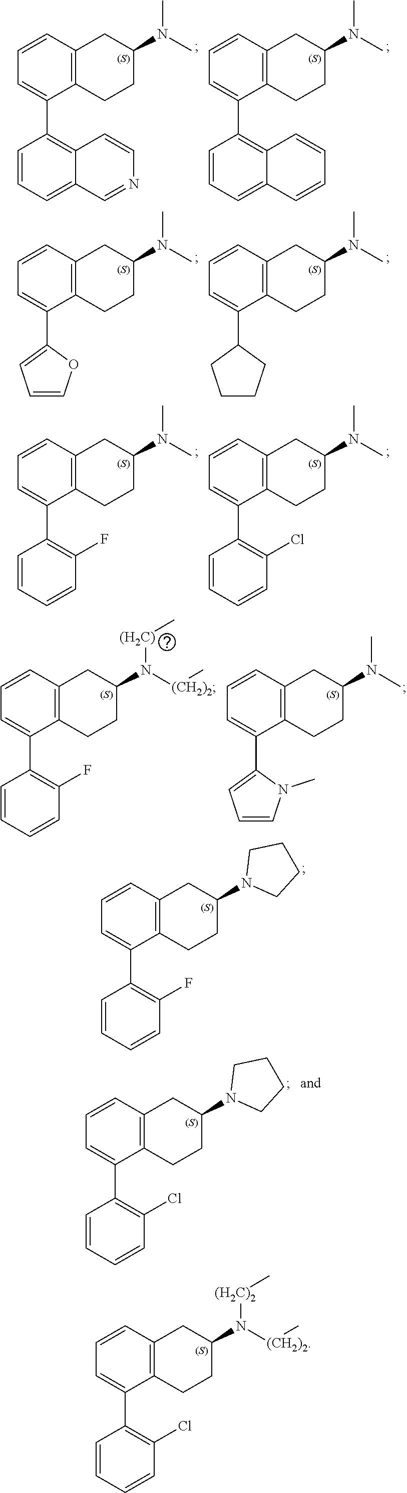 [chemical structure]
